(19) United States
(12) Patent Application Publication
(10) Pub. No.: US 2021/0225468 A1
(43) Pub. Date: Jul. 22, 2021

(54) SYSTEMS, DEVICES, AND METHODS FOR STANDARDIZING A FORMAT FOR MEDICAL INFORMATION RECEIVED FROM A PLURALITY OF SOURCES, ASSOCIATING THE STANDARDIZED MEDICAL INFORMATION WITH PATIENT ACCOUNTS STORED IN A PATIENT ACCOUNT DATABASE, AND PROVIDING ACCESS TO THE PATIENT ACCOUNT DATABASE VIA MEDICAL PORTAL INTERFACES

(71) Applicant: OutcomeMD, Inc., Los Angeles, CA (US)

(72) Inventors: Justin SALIMAN, Los Angeles, CA (US); April MILLER, Los Angeles, CA (US); Jason HURST, Vancouver, WA (US); Ryan SALIMAN, Los Angeles, CA (US); Karthik KARUNANITHI, Los Angeles, CA (US); Douglas GRIM, Portland, OR (US)

(21) Appl. No.: 17/155,034

(22) Filed: Jan. 21, 2021

Related U.S. Application Data
(60) Provisional application No. 62/963,858, filed on Jan. 21, 2020.

(51) Int. Cl. G16H 10/60; G16H 10/20; G06F 16/245; G06F 16/25; G16H 50/30

(52) U.S. Cl. CPC G16H 10/60; G16H 10/20; G16H 50/30; G06F 16/258; G06F 16/245

(57) ABSTRACT

Medical information, such as diagnosis, treatments provided, age, patient compliance with treatment, wellness scores, and/or improvement scores, for a plurality of patients received from a plurality of sources in a respective plurality of formats may be translated into standardized and associated with patient accounts for the respective plurality of patients and stored in a patient account database. The patient account database may be searchable for information on patients associated with the patient accounts.

### RELATED APPLICATION

The present application is a NON-PROVISIONAL, of and claims priority to, U.S. Patent Application No. 62/963,858, filed on 21 Jan. 2020 entitled “Systems, Devices, And Methods for Generating and Displaying Filtered Medical Portal Interfaces,” which is incorporated, in its entirety, herein.

### FIELD OF THE INVENTION

The present invention relates to medical information technology. More specifically, the present invention relates to systems and methods for standardizing a format for medical information received from a plurality of sources in a respective plurality of formats and associating the standardized medical information with patient accounts stored in a patient account database, the patient account database being searchable for information on patients associated with the patient accounts.

### BACKGROUND

Current medical and healthcare systems lack universal outcome measures and ways to access and/or analyze these universal outcome measures to search for patterns and/or determine possible and/or likely outcomes for patients prior to delivering treatment to them. Current medical and healthcare systems are also unable to accommodate and/or incorporate information from various sources to develop a larger picture of patient health and/or behavior due to incompatibility of the medical and healthcare systems and other sources of information.

### SUMMARY

Systems and methods for standardizing a format for medical information received from a plurality of sources in a respective plurality of formats and associating the standardized medical information with patient accounts stored in a patient account database, the patient account database being searchable for information on patients associated with the patient accounts are disclosed herein. The patient account database may include information regarding, for example, a patient, his or her health, wellness scores, improvement scores, treatments received, compliance with a proscribed treatment, demographic information, comorbidities, outcomes from a treatment that are tracked or scored over time. In some embodiments, the patient account database may be populated with patient responses to one or more medical questionnaires and/or outcome measurement devices (OMDs) such as test results, assessments, and clinical observations that are associated with the respective patients. The responses to the medical questionnaires and/or OMDs may be scored using one or more scoring procedures that are, in most cases, specific to the medical questionnaire. The scored questionnaires may be used to generate a wellness score, which in some cases may be normalized on a scale (e.g., 1-100 or 1-10). In some instances, a wellness score may be a composite of responses to multiple medical questionnaires with each medical questionnaire being scored with its respective scoring procedure. In these instances, normalizing the scored medical questionnaire responses may include aggregating the multiple scores of the medical questionnaires to form an overall wellness score that indicates a level of wellness for the patient across multiple medical questionnaires.

In some embodiments, patient information, responses to medical questionnaires, etc. may be de-identified (e.g., replacing patient name and/or demographic information with, for example, a binary, alphanumeric, and/or encrypted code) so that the patient account database may be accessed and/or searched by individuals/users who may not be authorized to view identifying information pertaining to a particular patient or group of patients. In this way, the data stored in patient account database may be accessed and/or searched by, for example, third parties, medical professionals, and patients who are not authorized to view identity information for one or more patients who have data stored in a patient account and/or in patient account database.

In some embodiments, systems, devices, and/or methods disclosed herein may include providing a medical questionnaire and/or OMD to a patient associated with a patient account stored in the patient account database. The patient account may be associated with a plurality of characteristics for the patient and the medical questionnaire/OMD may pertain to, for example, a diagnosis for the patient and/or a treatment received by the patient. The medical questionnaire/OMD may be associated with metadata that, for example, identifies the patient, includes information that may be used to de-identify the patient (e.g., a code), a treatment facility who triggered provision of the medical questionnaire/OMD to the patient, a treatment provider who triggered provision of the medical questionnaire/OMD to the patient, and/or a treatment and/or diagnosis associated with the medical questionnaire/OMD.

A set of responses to the medical questionnaire may be received from the patient and a wellness score for the patient may be determined by, for example, applying a scoring procedure associated with the medical questionnaire/OMD to the set of responses. In some embodiments, the set of responses may be associated with metadata that, in some cases is similar to the metadata that may be associated with the medical questionnaire/OMD. The metadata associated with the set of responses may include, for example, patient information (which may be de-identified), treatment provider information, treatment facility information, treatment and/or diagnosis information.

The set of responses and the wellness score into a standardized format compatible with the patient account database, which may serve to generate a standardized set of responses and/or a standardized wellness score. The standardized set of responses and/or the standardized wellness score may then be associated with the patient account and one or more of the plurality of characteristics for the patient. The standardized set of responses, the standardized wellness score, and the associations between the standardized set of responses and the standardized wellness score with the patient account and the one or more plurality of characteristics for the patient may then be saved in the patient account database.

An electronic medical record (EMR) database may be queried for information about the patient and/or one or more different patients/patient accounts who are associated with one or more of the plurality of characteristics of the patient. For example, the EMR may be queried for wellness scores, treatment compliance information, improvement scores, comorbidities, test results, disease progression over time, and/or demographic information of patients who match one or more characteristics of a particular patient (e.g., the patient who has submitted the set of responses), have undergone a treatment administered, or under consideration for administration, to the particular patient and/or have a diagnosis in common with the particular patient.

Often times, the EMR database is, for example, an EMR database maintained by a medical facility (e.g., hospital or clinic) and the information stored on the EMR database is in a format specific to the medical facility and/or EMR databases. In these instances, formation of the query may include translating a request for information received from the EMR database received from a user into a form compatible with the EMR database.

The queried-for electronic medical record information may be received from the EMR database and translated into the standardized format thereby generating a standardized set of electronic medical information. The standardized set of electronic medical record information may then be associated with the patient account and the plurality of characteristics of the patient. These associations, as well as the standardized set of electronic medical record information saved in the patient account database.

A user may then be provided with access to the patient account database via a display device and/or user interface (e.g., touch screen or keyboard).

In some embodiments, additional information may be received from, for example, a medical professional (e.g., doctor, nurse, pharmacist, etc.) and/or medical service provider (e.g., transportation service, meal provision service, etc.). Exemplary additional information includes, but is not limited to, demographic information about the patient, a diagnosis of the patient, a treatment administered to the patient, a comorbidity of the patient, and a degree of the patient's compliance with a treatment. The additional information may be received as part of, for example, a medical examination or other encounter with the medical professional. In some embodiments, this information may be received directly by the patient account database and, in other instances, this information may be received by, for example, the EMR database. The received patient information may be translated into the standardized format thereby generating a standardized set of patient information that may be associated with the patient account. The standardized set of patient information and the association between standardized set of patient information with the patient account may then be stored in the patient account database and provided to the user.

In some embodiments, translation of the set of responses, the wellness score, and/or the received electronic medical record information about the patient into the standardized format includes analyzing at least one of the set of responses, the wellness score, and the received electronic medical record information about the patient to determine a characteristic thereof. A code (e.g., binary, alphanumeric, cryptographic) may then be assigned to the respective set of responses, wellness score, and/or received electronic medical record information about the patient responsively to their respective determined characteristics. In some cases, the assigning may include associating the assigned code with the set of responses, wellness score, and/or received electronic medical record information and the saving may include saving the assigned code with the respective set of responses, wellness score, and/or received electronic medical record information.

Additionally, or alternatively, translating the set of responses, the wellness score, and/or the received electronic medical record information about the patient into the standardized format may include removing any patient-identifying information therefrom. At times, this process may also include association of a code or anonymous identifier with the set of responses, the wellness score, and/or the received electronic medical record information.

In some embodiments, each of the plurality of characteristics may be associated with a code and the association of the plurality of characteristics with the standardized set of responses and the standardized wellness score with the patient account may be performed by associating a code for each respective characteristic with the standardized set of responses and the standardized wellness score.

In some embodiments, the patient account database may store patient accounts for a large number (e.g., 1,000-10 million) patients and each of the patient accounts may be associated with a patient, a set of characteristics for each respective patient, and at least one wellness score determined for each respective patient. In these embodiments, a request for wellness scores and/or improvements scores of patients who match one or more specific characteristic(s) may be received and a query for the patient account database may be generated responsively thereto. The patient account database may then be queried for wellness scores of patients who match the specified characteristic(s) responsively to the received request and a set of wellness scores of patients who match the specified characteristic(s) may be received responsively to the query. The set of wellness and/or improvement scores may then be formatted for display on the display device and provided to the display device. For example, a user who is caring for a patient diagnosed with breast cancer may query the patient account database for wellness and/or improvement scores for patients who share one or more characteristics of the patient of interest such as age, gender, race, and treatment type.

In some instances, a plurality of treatments may be queried for so that wellness and/or improvement scores for patients who share the same characteristics (e.g., age, gender, race, and cancer type) but who received different types of treatment may be queried for in order to assess, for example, which may be the best treatment option for the patient. For example, a second query for wellness scores associated with patients that are associated with a second characteristic may be received and the patient account database may be queried for the requested information. A second set of wellness/improvement scores may then be received from the patient account database that match the second characteristic and formatted for display on the display device. The formatted second set of wellness/improvement scores may then be provided to the display device.

In some embodiments, the set of responses may be a first set of responses and the wellness score may be a first wellness score and the medical questionnaire may be provided to the patient a second, subsequent, time perhaps 1 week, 1 month, or 1 year following provision of the first medical questionnaire. A second set of responses to the medical questionnaire may be received from the patient and a second wellness score may be determined using the second set of responses. The first and second wellness scores may then be compared, and an improvement score may be determined based on the comparison. The second set of responses, the second wellness score, and/or the improvement score may then be translated into the standardized format compatible, thereby generating a second standardized set of responses, a second standardized wellness score, and a standardized improvement score, that may be associated with the patient account and the plurality of characteristics for the patient. The standardized second set of responses, the standardized second wellness score, the standardized improvement score, and the associations between the standardized second set of responses, the standardized second wellness score, the standardized improvement score with the patient account and the plurality of characteristics for the patient may then be saved in the patient account database.

In some embodiments, improvement scores may be determined for many (e.g., 1,000-10 million) patients and, in these embodiments, a request for improvement scores for patients that are associated with one or more specific characteristic(s) may be received. A query for the patient account database for improvement scores of patients associated with the specific characteristic(s) may be generated and communicated to the patient account database responsively to the request. A set of improvement scores that match the specific characteristic(s) may then be received from the patient account database responsively to the query, formatted for display on the display device, and provided to the display device.

In some embodiments, a request for information may be communicated to a third party source of information, such as a pharmacy, a transportation service, a home health care service, and/or a medical treatment provider and a set of third party information from the third party source of information may be received responsively to the request. At time the request regard the patient, the treatment, and/or the diagnosis. The set of third party information may then be translated into the standardized format thereby generating a standardized set of third party information, which may be associated with the patient account. The standardized set of third party information and the association between standardized set of third party information with the patient account may then be saved in the patient account database.

In some embodiments, a request for wellness scores associated with patients that are associated with a characteristic may be received. Exemplary characteristics include demographic information of the patients, a diagnosis of the patients, a treatment administered to the patient, a time following treatment being administered to the patient, patient compliance with treatment, and comorbidities of the patients. A patient account database may be queried for wellness and/or improvement scores of patients associated with the characteristic. The patient account database may store patient account information for a plurality (e.g., 1,000-10 million) patients and each patient account may be associated with at least one patient and wellness score determined for the patient responsively to receiving a set of responses to a medical questionnaire from the patient. A set of wellness scores that match the characteristic may be received from the patient account database responsively to the query, formatted for display on a display device, and provided to the display device. The formatting may include calculating an average wellness score for all patients who match the characteristic. In embodiments where an improvement score is requested, the request may further include a duration of time following initiation of a treatment administered to the patient, wherein a duration of time between an initially-determined and later-determined wellness score for the corresponds to the duration of time.

In some embodiments, a subsequent, or second request for wellness scores associated with patients that are associated with a second characteristic may be received and the patient account database may be queried for wellness scores of patients associated with the second characteristic. A second set of wellness scores that match the second characteristic may be received from the patient account database responsively to the query, formatted for display on a display device, and the formatted second set of wellness scores may be provided to the display device. In some cases, the second request narrows a scope of the first request. Formatting the second set of wellness scores may include calculating an average wellness score for all patients who match the first and second characteristics.

### BRIEF DESCRIPTION OF DRAWINGS

The present invention is illustrated by way of example, and not limitation, in the figures of the accompanying drawings in which:

FIGS. 1A and 1B are block diagrams of exemplary systems for generating and/or presenting a medical portal interface, in accordance with some embodiments of the present invention;

FIGS. 2A-C are screen shows of exemplary medical portal interfaces by which a user may select one or more assessments, or OMDs, to communicate to a patient, in accordance with some embodiments of the present invention;

FIGS. 3A and 3B provide a flowchart of a process for the generation of a medical portal interface that provides access to a patient-entered information, medical-treatment-provider-entered information, electronic medical record information, and third-party information that may be associated with one or more patient accounts, in accordance with some embodiments of the present invention;

FIG. 4 provides a flowchart illustrating a process for generating and providing for display of a medical information portal interface, in accordance with some embodiments of the present invention;

FIGS. 5A-5I are screen shots of exemplary interfaces showing various aspects of the medical portal interface, in accordance with some embodiments of the present invention;

FIG. 6 is a screen shot an exemplary outcome results overview interface page, in accordance with some embodiments of the present invention;

FIGS. 7A-7G provide screen shots of exemplary medical information portal interfaces, in accordance with some embodiments of the present invention;

FIGS. 8A-8E are screen shots showing a series of notification setting interfaces, in accordance with some embodiments of the present invention;

FIGS. 9A and 9B provide exemplary patient-specific interfaces, in accordance with some embodiments of the present invention; and

FIG. 10 is a block diagram showing a system, in accordance with some embodiments of the present invention.

### WRITTEN DESCRIPTION

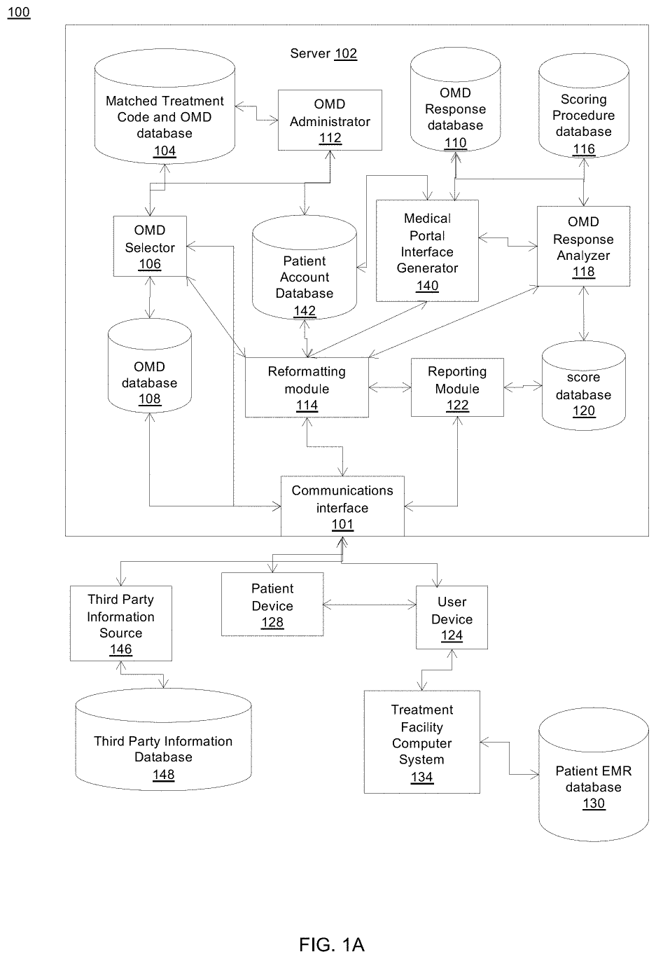

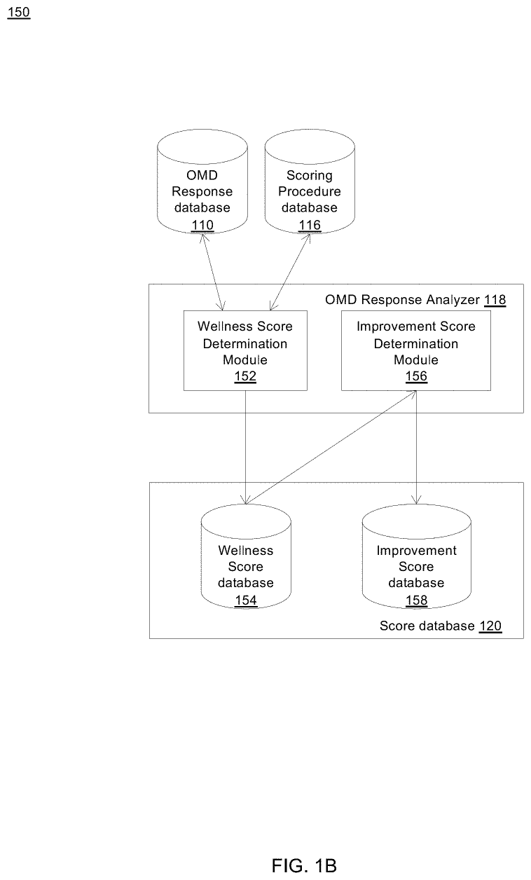

FIG. 1A provides a block diagram of an exemplary system 100 that may be used to execute one or more of the processes described herein. At a high level, system 100 includes a server 102, a patient device 128, a user device 124, and a treatment facility computer system 134, all directly or indirectly communicatively coupled to one. Patient device 128 may be any device (e.g., a smartphone, a laptop computer, a tablet computer, a desktop computer, etc.) that enables communication between a patient and other components of system 100. Similarly, user device 124 may be any device (e.g., a smartphone, a tablet computer, a laptop computer, a desktop computer, etc.) that enables communication between a treatment provider (also referred to herein as a “user”) and other components of system 100. In some cases, user device 124 may also be a device that is enabled to perform a specific healthcare treatment and/or diagnostic task. For example, user device 124 may be a network-connected treadmill, a network connected blood pressure monitor, or a network-connected ultrasound machine. For simplicity, only one user device 124 is depicted, while it is understood that in practice there may be a plurality of user devices, one or more for each treatment provider. Similarly, while only one patient device 128 is depicted, it is understood that in practice there may be a plurality of patient devices, one or more for each patient.

Treatment facility computer system 134 may be a computer system that is located in, and/or communicatively coupled to, a treatment facility (i.e., a computer/server that is located in a doctor's office or treatment facility). As is understood in the art, an EMR (as stored in EMR database 130) may include notes prepared by a treatment provider regarding the health of a patient, results of medical tests performed on a patient, treatments administered on a patient, etc. Further due to HIPAA regulations, medical records from treatment facility computer system 134 may be communicated to user device 124, patient device 128 and server 102 using one or more security protocols that may be compliant with HIPAA requirements. It is understood that other data (i.e., not patient-specific data) may be transmitted between user device 124, patient device 128, sever 102 and facility computer system 134 via a conventional communication network (e.g., the Internet, a wired network, a wireless network, a private network, a public network, routers, switches, etc.), which has not been depicted in FIG. 1A. Further, it is understood that user device 124, patient device 128, server 102 and facility computer system 134 may be communicatively coupled to via a communication network (e.g., the Internet) and/or a blockchain.

In one embodiment, any one of the components of system 100 may replace any patient identifying information (e.g., patient name, social security number, birthdate, address, etc.) in medical records with, for example, a binary string to form anonymized medical records containing no patient identifying information (e.g., patient name, social security number, birthdate, address, etc.). More generally, any patient identifying information in medical data (e.g., EMR, questionnaire responses provided by a patient, wellness scores computed for a patient, etc.) may be replaced with a binary string to form anonymized medical data. Such anonymized medical data may be stored at, for example, server 102, treatment facility computer system 134, patient device 128, and/or user device 124, in various databases operated by server 102 (e.g., OMD response database 110, score database 120, etc.), cloud-based storage (e.g., Amazon Web services, Google Cloud platform or Microsoft Azure) (not depicted), etc. In the event the anonymized medical data is intercepted by a malicious individual (e.g., a hacker), patient privacy may still be preserved since the malicious individual will not be able to associate the anonymized medical data with any specific patient.

A mapping between respective binary strings and respective patient identifying information may be securely stored (e.g., stored in an encrypted manner) at one or more components of system 100. Such mapping may enable an electronic device (e.g., server 102, user device 124, and/or patient device 128) to access medical data associated with a specific patient. The steps for an electronic device to access the medical data of a patient may proceed as follows. First, the electronic device may be authenticated by HIPAA compliance server (e.g., the electronic device is required to provide the proper credentials, such as a login identifier and password). Following successful authentication, the electronic device may request medical data concerning an exemplary patient, John Doe. For example, server 102 may map the patient name of “John Doe” to “patient 001010” via the mapping and/or indexing, and the medical data of patient 001010 may be retrieved from a database which stores the anonymized medical data (e.g., OMD response database 110, score database 120, etc.).

In one embodiment, the process flow for system 100 may proceed as follows. Upon server 102 receiving a request from, for example, user device 124 and/or patient device 128, server 102 may provide an outcome measurement device (OMD) to the patient and/or user device 124 and/or 128. An OMD may be a modality, instrument, or tool by which medical information about a patient may be collected. Exemplary OMDs include, but are not limited to, a medical questionnaire, a physical test of the patient (e.g., blood test, physical examination, or blood pressure), and a patient reported outcome (PRO) instrument. At times, an OMD may referred to as a medical questionnaire herein. In some cases, the request to administer the OMD may be triggered via the entry of a treatment code (e.g., a Current Procedural Terminology (CPT) code) or a treatment/diagnostic test name into the patient's EMR (as stored in patient EMR database 130), a treatment facility's billing software, and/or a treatment facility's scheduling software. In some instances, a request to administer and OMD may be triggered by a patient requesting receipt of an OMD via, for example, his or her wellness account and/or a request to administer and OMD may be triggered by a patient who requests to send an OMD to a friend or colleague via, for example, a link to an OMD and/or an invitation to respond to an OMD.

In some instances, when a treatment/diagnostic test name or other related information (other than a treatment/diagnostic code) is received, server 102 may interpret (using, for example, natural language analysis) the treatment/diagnostic test name so that it matches one or more treatment codes. In such cases, OMD selector 106 may determine one or more OMDs that match the treatment code via matched treatment code and OMD database 104. More generally, matched treatment code and OMD database 104 may also include matches between treatment names and OMDs, as well as diagnostic codes and OMDs when selecting OMDs for delivery to a patient device 128 and/or user device 124.

Next, OMD selector 106 may retrieve the one or more determined OMDs from OMD database 108. The retrieved OMDs may be provided to OMD administrator 112, which may administer the OMDs to the patient via, for example, patient device 128 and/or user device 124. In the instance that the retrieved OMDs are patient reported outcome (PRO) instruments, the PRO instruments may be provided to patient device 128. Completed OMDs (also called OMD responses) may be transmitted from patient device 128 and stored in OMD response database 110. More specifically, OMD responses may be stored in OMD response database 110 in an anonymized fashion. For example, OMD responses may be indexed in OMD response database 110 by a binary string, or other anonymous identifier, rather than by a patient name. Similarly, to the discussion above, if an OMD response for a specific patient is desired, the patient name may be mapped to a binary string by, for example, server 102, and the OMD response associated with that binary string may be retrieved from OMD response database 110.

OMD response analyzer 118 may analyze the OMD responses stored in OMD response database 110 to generate one or more scores (e.g., a wellness score, an improvement score, etc.). Such scores are described in more detail below with regard to FIG. 1B. Such analysis may rely upon scoring procedures stored in scoring procedure database 116. Such scoring may also rely upon other considerations and/or esoteric factors 132 stored at patient EMR database 130. In most circumstances, what may be referred to herein as “other considerations” are factors that may directly, or closely, relate to and/or have an impact on, a medical condition, diagnosis, and/or treatment. For example, it is known that smoking has an impact on a person's cardiovascular health. Thus, whether a person smokes may be an “other consideration” for a patient's treatment related to cardiovascular health. This relationship between cardiovascular health and smoking may be indexed or otherwise stored in other consideration database 132. An esoteric factor is one that is less directly related to a medical condition, diagnosis, and/or treatment but may still have an impact thereon. For example, a vegetarian diet may have an impact on a person's cardiovascular health, yet this impact may be less well understood when compared with the impact of smoking on the same patient's cardiovascular health. As such, a person's status as a vegetarian may be considered an “esoteric factor.”

The scores that are generated by OMD response analyzer 118 may be stored at score database 120. More specifically, scores may be stored in score database 120 in an anonymized fashion so as to, for example, comply with HIPAA regulations or other data security requirements/preferences. For instance, wellness scores associated with a patient may be indexed by a binary string in score database 120 rather than by a patient name.

Finally, reporting module 122 may report the scores to one or more of user device 124, patient device 128 and treatment facility computer system 134. In addition to the request for a treatment, there are other events that may prompt an OMD to be administered to a patient. In one example, the scheduling of an initial appointment (e.g., a consultation) for a patient to discuss a medical condition with a healthcare professional may prompt an OMD to be administered to the patient. Administering an OMD to the patient prior to this initial appointment may be useful for establishing a baseline state of health for the patient, but the selection of the OMD may have some complexity, as no treatment code, treatment name or diagnostic code may yet be available when the initial appointment is scheduled. In many instances, all that the patient will provide is a brief description of the symptoms he/she may be experiencing (e.g., shortness of breath, fever, etc.) and/or a chief complaint. In one embodiment, such symptoms may be provided to OMD selector 106, which attempts to match the symptoms with one or more treatment codes, treatment names, or diagnostic codes.

Such matching by OMD selector 106 may be performed using a learning machine. For instance, matches between, for example, symptoms and treatment codes; symptom and treatment names; and/or symptoms and diagnostic codes) may be provided by a healthcare professional when treating patients, and such matches may be used to train a model that can then be used to determine treatment codes, treatment names or diagnostic codes based on, for example, a patient's symptoms and/or treatment provider notes. Upon determining a treatment code, treatment name, or a diagnostic code, OMD selector 106 may select one or more OMDs based on matches provided in matched treatment code and OMD database 104 (as described above). It is anticipated that the determination of a treatment code, treatment name or diagnostic code by OMD selector 106 may be, in some instances, an imperfect process, so a healthcare provider, or other expert, may be asked to make any necessary adjustments to the treatment code, treatment name and/or diagnostic code determination, before OMD selector 106 selects the one or more OMDs.

In the examples provided above, it was assumed that an OMD is administered to a patient via patient device 128. In other instances, a medical professional may be required to administer the OMD to the patient. For example, server 102 may notify user device 124 that one or more OMDs should be administered as part of, for example, a medical examination of a patient. In one example, if a patient has recently undergone cardiothoracic surgery, OMD administrator 112 may provide one or more OMDs to user device 124 (e.g., the Intrathoracic Gas Volume Test, Total Lung Capacity Test, Vital Capacity Test, 6 Minute Walk Test, Aortic Insufficiency Test, Mitral Regurgitation Test and/or Aortic Valve Area Test) that could, or should, be administered to the patient during an exam and/or provide one or more mechanisms to user device 124 (e.g., fillable forms) for the treatment provider to enter the OMD responses.

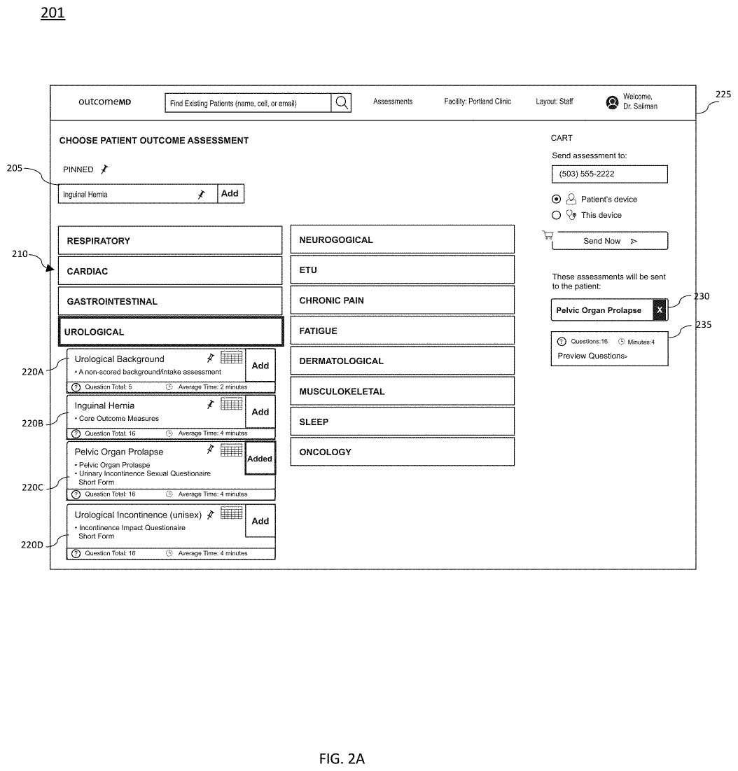

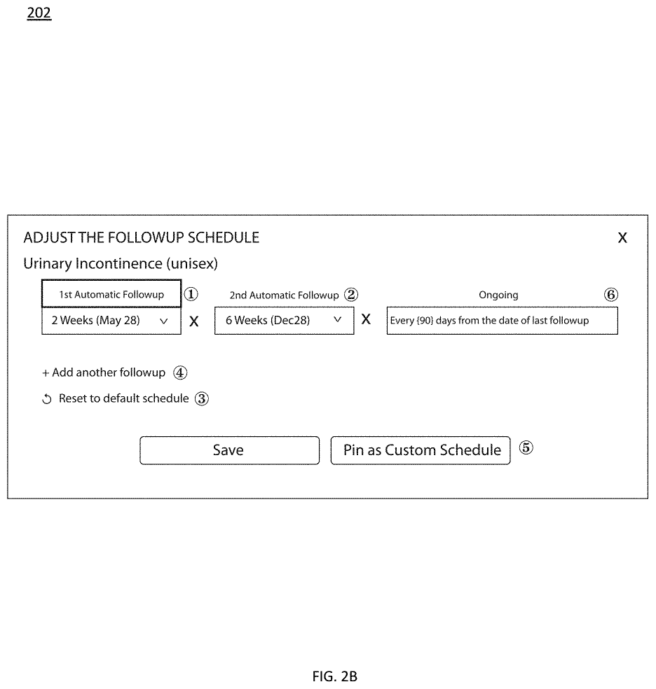

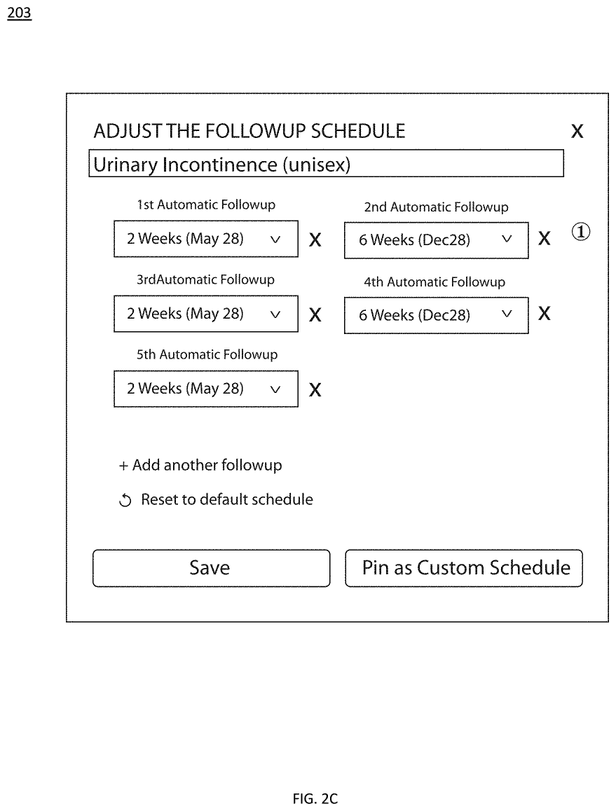

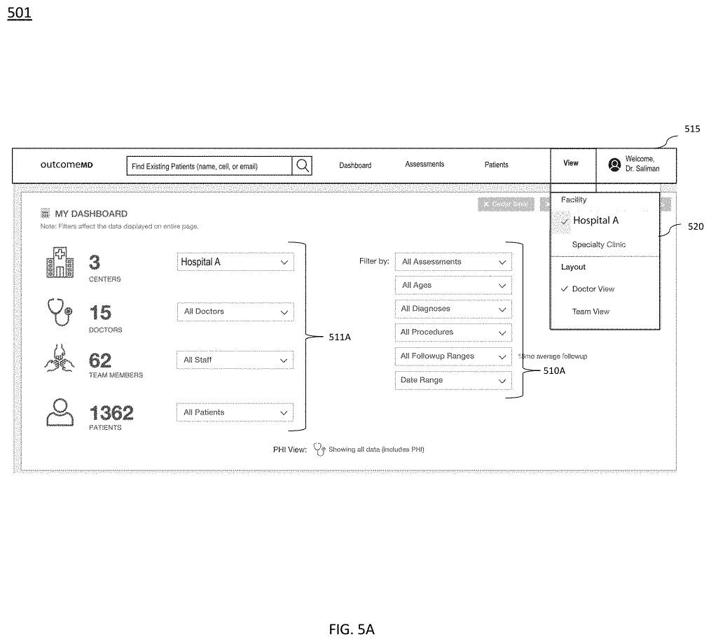

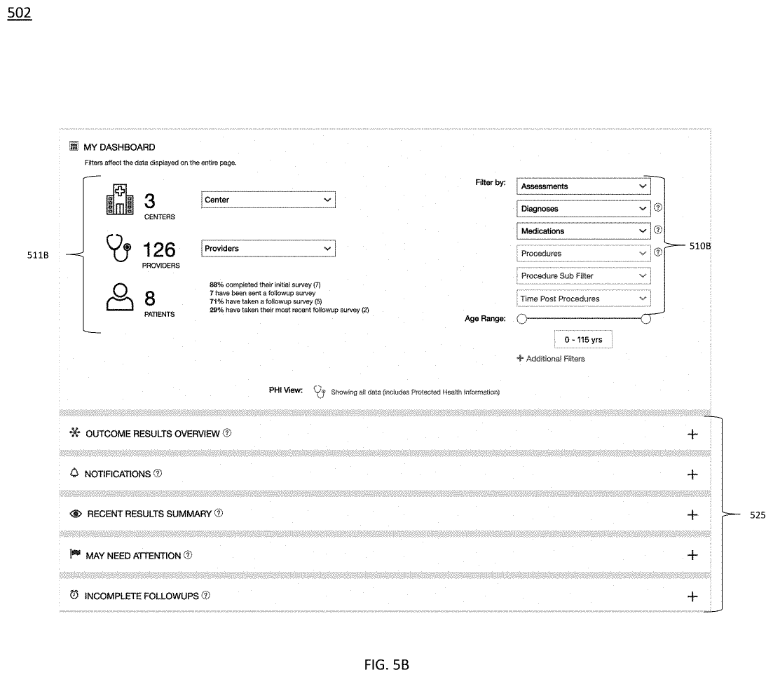

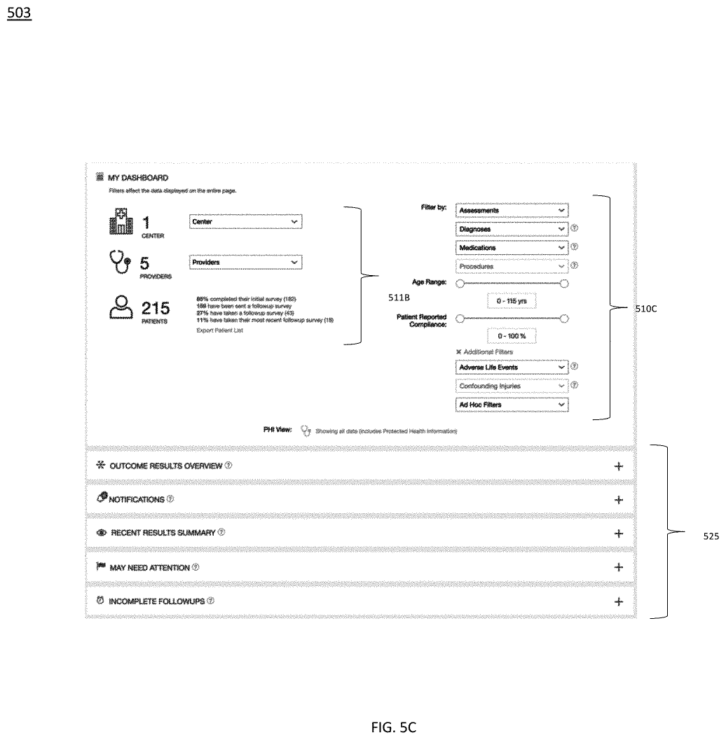

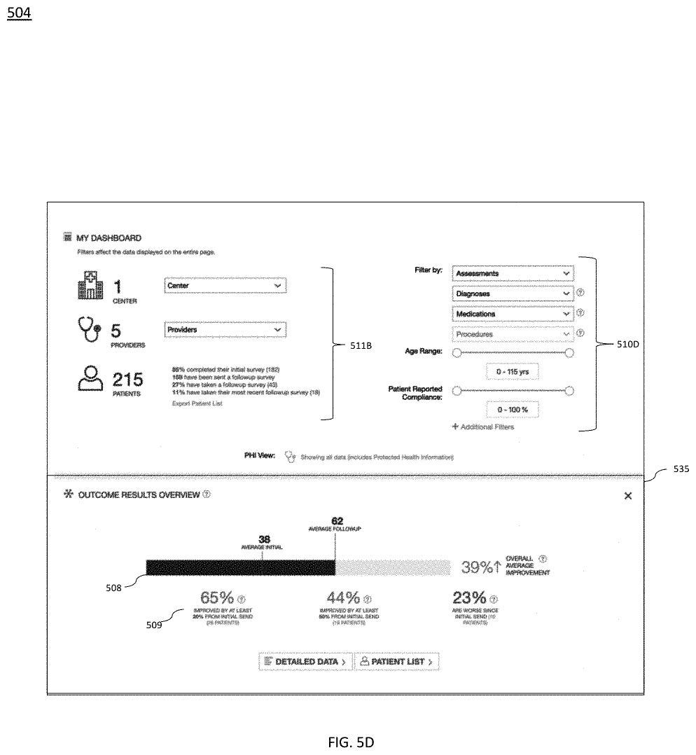

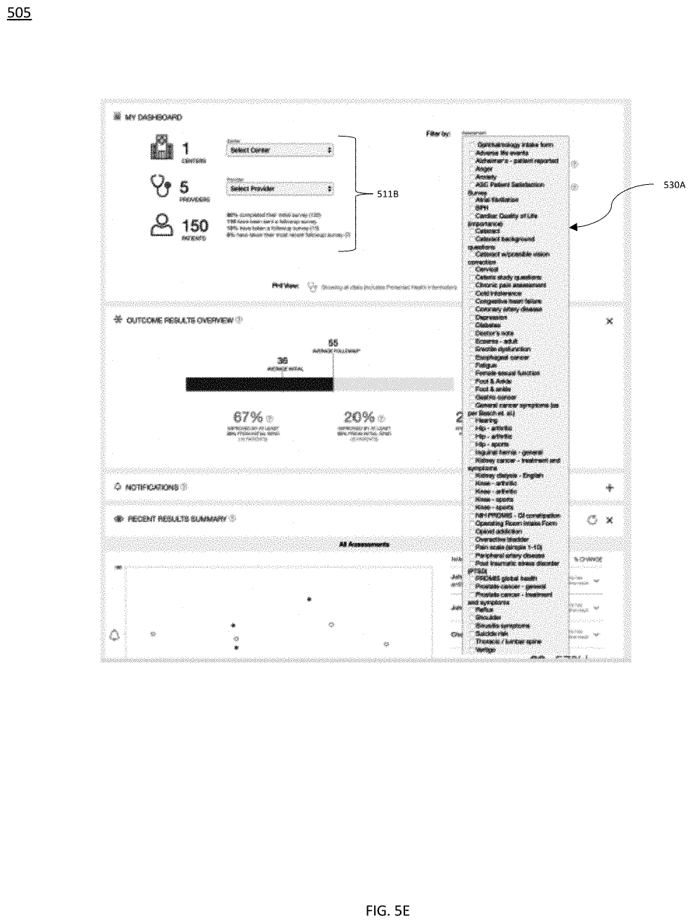

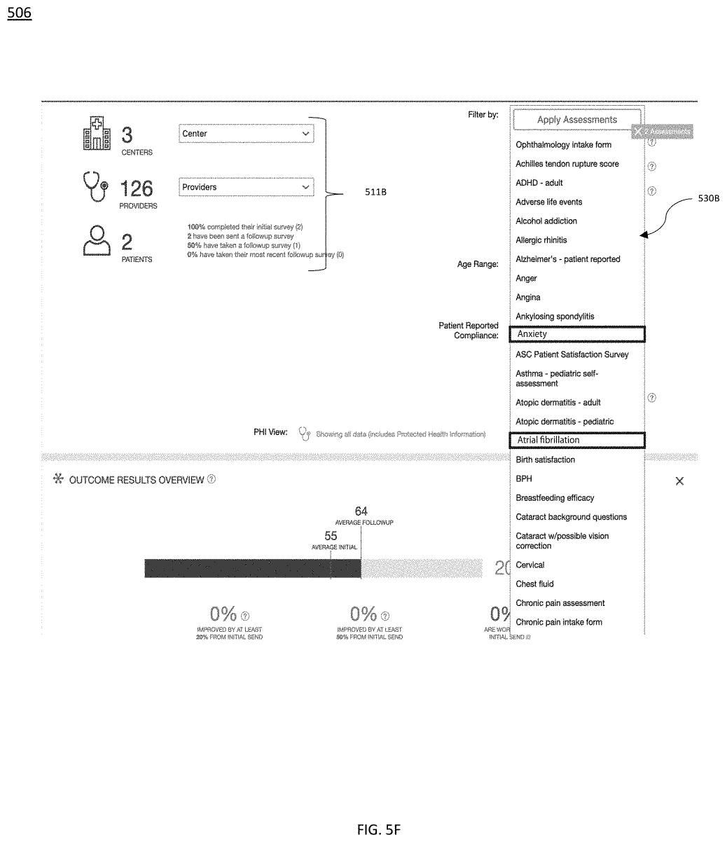

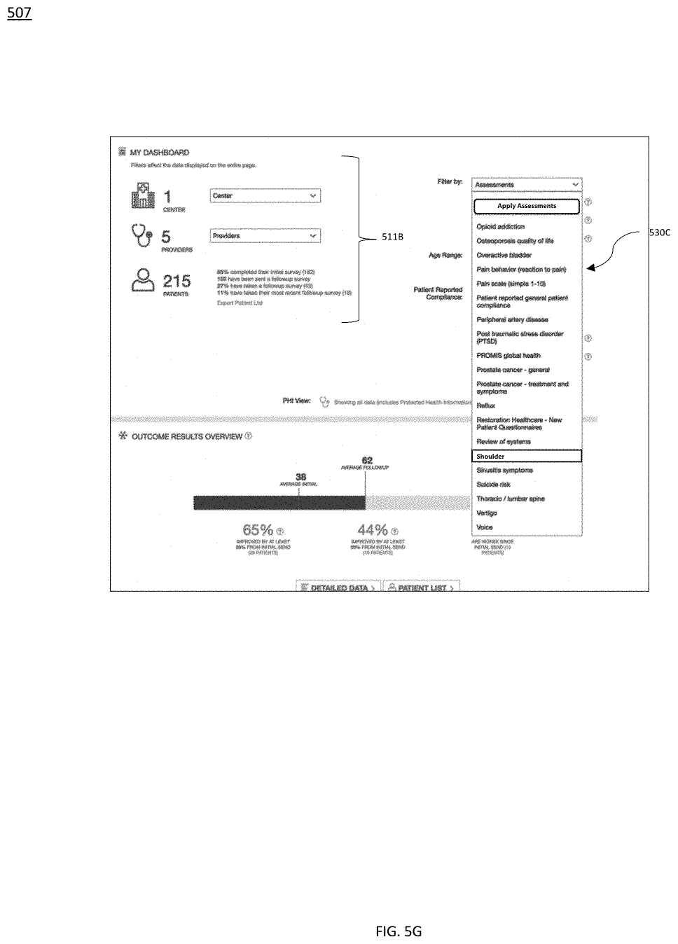

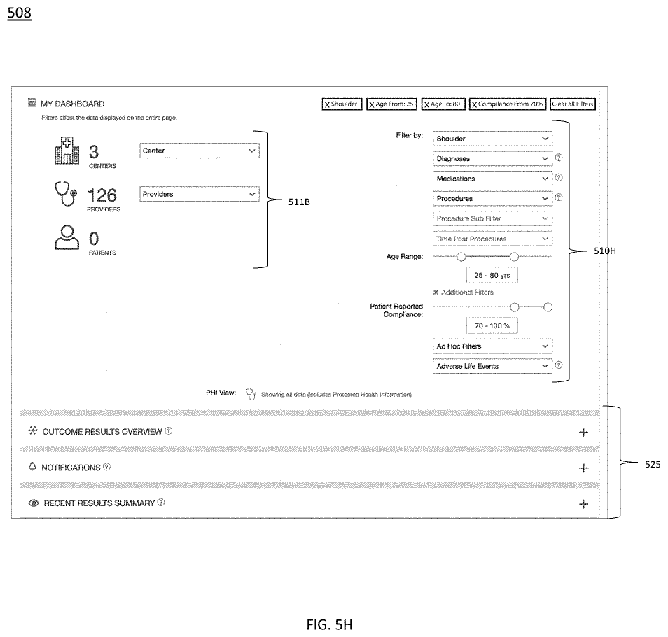

Server 102 may further include a patient account database 142, a medical portal interface generator 140, a reformatting module 114, a communications interface 101, a third-party information source 146, and a third-party information database 148. Communication interface 101 may facilitate communication between server 102 and an external device such as third-party information source 146, patient device 128, and/or user device 124. In some embodiments, communication interface 101 may resemble communication interface 1118. Medical portal interface generator 140 may generate one or more medical portal interfaces, like the medical portal interfaces shown in FIGS. 2A-C and FIGS. 5A-10B, using information received by and/or stored in server 102. For example, medical portal interface generator 140 may receive information from a user, patient, and/or third-party information source via, for example, user device 124, patient device 128, and/or third-party information source 146. The received information may be reformatted from an original and/or intermediate format into a format compatible with, for example, server 102 and/or medical portal interface generator 140 by reformatting module 114. The received information may then be added to and/or used to generate a medical portal interface by medical portal interface generator 140. Additionally, or alternatively, medical portal interface generator 140 may receive information from patient account database 142 (e.g., in response to a query sent by the medical portal interface generator 140 to patient account database 142) that may be used to add to and/or generate a medical portal interface by medical portal interface generator 140. Exemplary information that may be received from patient account database 142 includes, but is not limited to, patient identifiers, demographic information for one or more patients, patient-entered information (e.g., adverse life events, medication/treatment compliance, answers to OMDs, supplemental treatments, etc.), treatment history, historical OMD responses, wellness scores, improvements scores, and so on.

Third-party information source 146 may be any source of information that is not the patient and/or operated by and/or directly associated with a user of user device 124 (e.g., treatment facility staff or medical professionals) and/or an entity operating server 102. Examples include pharmacies, medical treatment facilities other than medical treatment facilities coupled to treatment facility computer system 134 and/or patient EMR database 130 (e.g., laboratories, radiologists, chiropractors, physical fitness facilities, etc.), medical device retailers, and other service providers for patients such as ride-share services that may be able to provide information regarding pick-up and drop-off times for patients at a facility that may administer medical treatment. Third-party information database 148 may store information relevant to one or more patients who may, or may not, have a patient account that may be stored in patient account database 142. Exemplary information stored in third-party information database 142 includes, but is not limited to, pharmaceutical refill information (e.g., dates refills were dispensed, type and/or quantity of pharmaceuticals dispensed, etc.), purchases made at/via the third-party information source 146 (e.g., supplements, braces, durable medical goods, etc.), clinic information (e.g., when appointments were scheduled, whether patient arrived for appointment, notes from a treatment provider regarding an encounter with a patient, etc.) and so on.

In some cases, medical portal interface generator may pull together information from various third-party information sources to build a complete picture of the health and/or treatment of one or more patients so that it may be conveyed via a medical portal interface like the medical portal interfaces of FIGS. 2A-2C and 5A-10B for analysis and study by a user.

Patient accounts may be associated with each individual patient under the care of a particular treatment facility. Information about a patient may be associated with and/or stored along with patient account information. Information about a patient may come from a plurality of sources including, but not limited to, the patient, a treatment provider, a user of a server providing access to a patient account, and third-party.

Patient accounts may be generated at/by server 102 responsively to instructions from the patient (as provided via, for example, patient device 128) and/or responsively to a user like a treatment facility administrator or medical treatment provider providing instructions via, for example, user device 124. Often times, the patient account is not directly linked (e.g., can receive information from and/or provide information to) all medical treatment and/or care providers. The medical treatment and/or care providers not in direct communication with a patient account

FIG. 1B depicts one embodiment of a system 150 that supports the operation of OMD response analyzer 118 and score database 120 (and some associated components). OMD response analyzer 118 may comprise wellness score determination module 152. In one embodiment, wellness score determination module 152 retrieves responses to an OMD from OMD response database 110, and further may retrieve a scoring procedure associated with the OMD responses from scoring procedure database 116. The scoring procedures may be indexed by, for example, an identifier of an OMD, for which responses have been received, making for easy retrieval of a corresponding scoring procedure. Various scoring procedures may be employed to score a completed OMD, and in one embodiment, the generated score may be known as a “wellness score”. In some cases, a “wellness score” may serve to indicate how severe a patient's symptoms are. In these cases, a low wellness score may indicate that a patient's symptoms are relatively more severe than a higher wellness score such that a subsequent higher wellness score indicates an improvement (i.e., decrease in severity) in the symptoms.

In the case where an OMD is a questionnaire (or PRO instrument), a certain weighing may be used to score or evaluate the patient's responses. For example, certain responses that are more objective in nature (e.g., heart rate, blood glucose level, etc.), may receive greater weights (and hence have a greater influence on the wellness score) than certain responses that are more subjective in nature (e.g., degree of pain, mood, etc.). The reverse scenario, of course, could be true in which subjective responses receive a greater weight than objective responses (e.g., fatigue or mental illness). Scores generated by wellness score determination module 152 may be stored in wellness score database 154. The wellness scores may be indexed in various fashions, for ease of retrieval. In one embodiment, wellness scores may be indexed according to one or more of a patient identifier (e.g., binary string to protect patient privacy), medical condition, treatment provider, treatment facility, time at which OMD was completed, etc.

Improvement score determination module 156 may retrieve two wellness scores for a patient (e.g., a first score calculated for an OMD completed at a first time point and a second score calculated for an OMD completed at a second time point) from wellness score database 154. Improvement score determination module 156 may calculate the difference between the first and second score, and such difference may be known as an improvement score. The improvement score may be stored in improvement score database 158. In one refinement, a relative improvement score may be calculated as the improvement score (i.e., the difference described above) normalized by a maximum improvement score, which may be calculated based on, for example, other considerations 132 stored in a patient's EMR. The maximum improvement score may take into consideration other factors such as the state of a patient prior to a medical treatment (e.g., if patient was in fairly good health, the maximum improvement score might be lower than if the patient was in poor health), and/or the age of a patient (e.g., younger patients might have a higher maximum improvement score than older patients), etc. An improvement score (or a relative improvement score) may be stored in improvement score database 158. The improvement scores may be indexed in various fashions, for ease of retrieval. In one embodiment, improvement scores may be indexed according to one or more of a patient identifier (e.g., binary string to protect patient privacy), medical condition, treatment provider, treatment facility, and time duration over which improvement score was measured, etc.

The components and/or databases of systems 100, 150, and/or 103 of FIGS. 1A, 1B, and/or 1C may be a series of one or more components (e.g., computers, servers, databases, etc.) that may, in some instances, be geographically disparate.

As disclosed herein, a wellness score and/or an improvement score for one or more aspects of the patient's medical condition and/or physiological systems may then be determined by scoring responses to one or more assessments, which in some cases may be patient reported outcome (PRO) assessments that have been validated to assess a patient's medical condition via medical literature and/or accepted best practices within the medical field. In some embodiments, determination of a wellness score may include querying a scoring database like scoring procedure database 116, for a scoring metric and/or scoring procedure associated with the medical questionnaire provided in step 205. In some instances, this querying may include retrieving a scoring procedure from scoring procedure database 116 using an identifier of the medical questionnaire. For instance, a medical questionnaire may be associated with a code (e.g., 3232) and this code may be used to retrieve a scoring procedure from scoring procedure database 116. Example scoring procedures include taking an average of all the patient responses (e.g., assuming all responses are numeric), taking a weighted average of the patient responses (e.g., weighting certain responses higher than other responses), adjusting the range of patient responses (e.g., changing responses choices from 2, 3, 3 to 1, 4, 6). In some embodiments, determining a wellness score may include retrieval of a sub-scoring procedure that may be specific to the patient (i.e., associated with the patient's account and/or a co-morbidity of the patient) as may be indicated by, for example, the patient's account and/or EMR. The scored responses may then be used to determine a wellness score associated with the received responses and/or a sub-set of received responses.

An improvement score (or percentage change) may be a determination of how a patient's condition has changed over time. In some embodiments, determination of an improvement score may involve comparing (e.g., averaging, subtracting, determining a percentage change, determining a time weighted average, etc.) one or more previously determined wellness scores with a currently determined wellness score in order to determine how a patient's wellness score has changed over time (e.g., 3 weeks, 3 months, 1 year, etc.).

FIGS. 2A-2C provide medical portal interfaces 201-203 by which a user may select one or more assessments, or OMDs, to communicate to a patient via, for example, patient device 128. For example, medical portal interface 201 of FIG. 2A includes a text box showing a diagnosis (in this case, inguinal hernia) and a menu of physiological systems, or categories, of OMDs. In the example of FIG. 2A, the urological system is selected and, responsive to its selection, a plurality of icons 220 representing OMDs that are associated with the selected system are displayed. Icons 220 provide an opportunity for user to add and OMD to a list of OMDs to be communicated to the user following completion of inputting information into interface 201 and/or calendaring the delivery of the OMD for another time. In this case, an icon representing an OMD for urological background 220A, an icon representing an OMD for inguinal hernia 220B, an icon representing an OMD for pelvic organ prolapse 220C, and an icon representing an OMD for urinary incontinence (unisex) 220D are provided. Of the OMDs available and add icon for pelvic organ prolapse icon 220C has been selected, or added, to a list of OMDs to be provided to a patient as shown list 230 of OMDs to be sent to a patient. Interface 201 also provides a menu 225 of options for a user to enter information and/or select an option for communication of the OMD to the patient along with an icon 235 providing an option to preview the questions of an OMD.

FIG. 2B provides an interface 202 by which a user may set and/or adjust a schedule (e.g., a follow up schedule) for the communication of OMDs that may be selected via an interface like interface 201 to a patient. For example, interface 202 provides a user with an opportunity to set first and second automatic follow-up communications of OMDs to the patient, and add another follow-up to the patient's account icon, and/or an option to reset a delivery schedule to default settings.

FIG. 2C provides another interface 203 by which a user may set and/or adjust a schedule (e.g., a follow up schedule) for the communication of OMDs that may be selected via an interface like interface 201 to a patient. For example, interface 203 provides a user with an opportunity to set first-fifth automatic follow-up communications of OMDs to the patient, and add another follow-up to the patient's account icon, and/or an option to reset a delivery schedule to default settings.

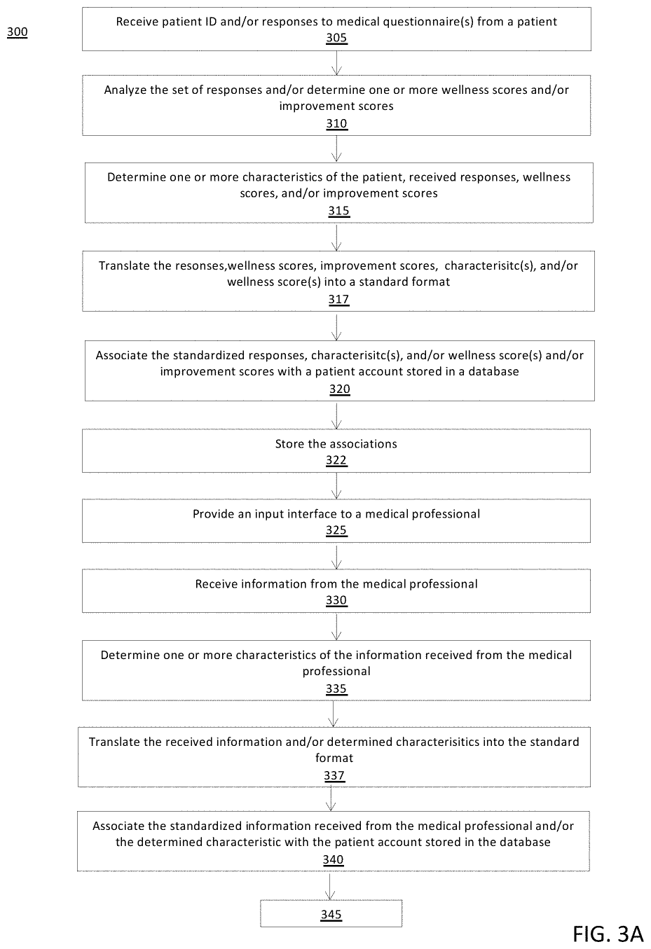

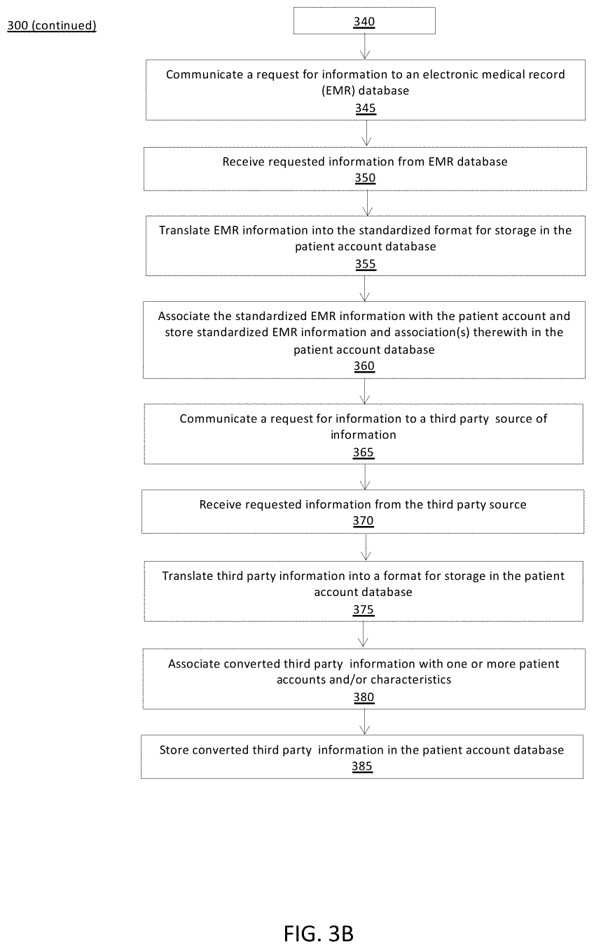

FIGS. 3A and 3B provide a flowchart of a process 300 that standardizes information about a patient received from a plurality of different sources in a respective plurality of different formats into a standardized format and associates the standardized information with a patient account for the patient. Process 300 may be executed by, for example, systems 100 and/or 101 and/or components thereof. Exemplary information about patients includes, but is not limited to, patient-entered information, responses to medical questionnaires, medical-treatment-provider-entered information, electronic medical record (EMR) information, and third-party information.

Process 300 may be executed a plurality (e.g., hundreds, thousands, millions) of times for a plurality of patients (e.g., hundreds, thousands, millions) so that, for example, a patient account database with standardized information about the plurality of patients may be generated and continuously updated. The patient account database may be searchable by users via, for example, one or more medical portal interfaces that provides access to a patient-entered information, medical-treatment-provider-entered information, electronic medical record (EMR) information, and third-party information that may be associated with one or more patient accounts.

Initially, a patient identifier and/or responses to a medical questionnaire may be received by, for example, a server like server 102 from a patient via, for example, a patient's personal electronic device such as patient device 128 and/or a user device, such as user device 124 (step 305). The medical questionnaire may be an OMD that may be communicated to the patient responsively to an instruction received from a treatment provider or other care giver via, for example, interfaces 201-203 discussed above with regard to FIGS. 2A-2C.

The responses may be entered into the electronic and/or user device by, for example, the patient and/or a medical treatment provider or care giver for the patient. The medical questionnaire may be or more OMDs. In some embodiments, the responses may be in a free text format where a patient provides a written narrative response to a question. At times, the medical questionnaire and/or OMD and/or responses thereto may pertain to how compliant a patient has been with a treatment regime and/or recovery plan.

The patient identifier may be, for example, identification information (e.g., username and password, patient-specific identification code, etc.) pertaining to a patient account within, for example, a patient account database like patient account database 142. The patient identifier may be used to associate information received via process 300 with the patient's account. In some cases, the patient identifier may be a code (e.g., a binary string or encrypted code) used in place of other identifying information to associate information received via process 300 with the patient's account so that, for example, patient information and/or patient account information may be anonymous and/or de-identified. In some embodiments, the patient identifier may be used to encrypt information received from the patient and/or responses received in step 305 so that, for example, the responses may be communicated from the patient's device to a server or other computer receiving the responses in step 305 in an encrypted and/or de-identified format.

In step 310, the set of responses may be analyzed to determine one or more characteristics thereof (step 315) and/or determine a wellness score therefrom. The wellness score may indicate how well the patient is doing with regard to one or more medical conditions he or she is diagnosed with. In some embodiments, determination of a wellness score may include querying a scoring database like scoring procedure database 116, for a scoring metric and/or scoring procedure associated with the medical questionnaire. In some instances, this querying may include retrieving a scoring procedure from scoring procedure database 116 using an identifier of the medical questionnaire. For instance, a medical questionnaire may be associated with a code (e.g., 3232) and this code may be used to retrieve a scoring procedure from scoring procedure database 116. Example scoring procedures include taking an average of all the patient responses (e.g., assuming all responses are numeric), taking a weighted average of the patient responses (e.g., weighting certain responses higher than other responses), adjusting the range of patient responses (e.g., changing responses choices from 2, 3, 3 to 1, 4, 6).

Additionally, or alternatively, in step 315, a characteristic of the patient (e.g., age, gender, diagnosis, attending physician, treatment recommendation, treatment facility, etc.) and/or the received responses may be determined. In some circumstances, these characteristics may be determined using an identifier of the patient and/or the medical questionnaire that may be received along with the responses in step 305. In some cases, performance of step 310 and/or 315 may include a determination of an improvement score for the patient. An improvement score may be determined by, for example, comparing two wellness scores; typically, two wellness scores determined using the same medical questionnaire but separated by a duration of time (e.g., weeks, months, years) to see how the wellness scores have changed over time. Improvement scores may be represented as, for example, a numerical value or a percentage.

In step 317, the responses, wellness scores, improvement scores, and/or patient characteristics, may be translated into a standardized format compatible with a receiving component (e.g., server) and/or a database coupled thereto like patient account database 142. In some embodiments, step 317 may be performed prior to execution of step 310 and/or 315 so that, for example, the wellness and/or improvement scores may be determined using standardized responses to the medical questionnaire/OMD and/or the responses, the wellness scores, and/or the improvement scores may be quantified and/or associated with the patient account and/or patient characteristics. Execution of step 317 may include encoding responses to the medical questionnaire/OMD, wellness scores, improvement scores, and/or patient characteristics, with, for example, one or more diagnostic and/or treatment codes, encryption, and/or translation of, for example, responses received in a user interface markup language or extensible markup language (XML) into a software language compatible with a database language like structured query language (SQL).

In some embodiments, translation of some of the responses, such as free-form text responses may include scanning the free-form text for keywords or quantifiable metrics so that the free-form text responses may be translated into a format that is standardized across patients and/or other contributors to information stored in the patient account database.

In step 320, the responses, characteristic(s), wellness scores, and/or improvement scores may be associated, correlated, or otherwise cross-referenced with a patient account, one or more characteristics of the patient, the medical questionnaire, a diagnosis of the patient and/or a treatment the patient has, or is, receiving. These associations may then be saved, or otherwise stored, in a database such as patient account database.

Optionally, in step 325, an input interface may be provided to a user (e.g., medical professional) by which the user may input information regarding the patient. The input interface may be provided to the user via his or her user device, which may be a user device like user device 124. Information input into the interface, or otherwise communicated, may be received in step 330 and may be translated into the format compatible with, for example, a receiving server (e.g., server 102) and or a database like patient account database 142 (step 337). In some cases, execution of step 317 may be similar to execution of step 337. Information input into the interface or otherwise received in step 325 may include information about the patient (e.g., notes from an examination of the patient (e.g., chief complaint, side effects, responsiveness to a treatment, etc.), demographic information, treatment administered to and/or proscribed for the patient, comorbidities of the patient, patient's compliance with a treatment, and so on. Translation of the information received in step 330 may include, but is not limited to, scanning free-form text for keywords that may be associated with, for example, standardize terms or codes so that the patient's condition or characteristics may be extracted from the free-form text and associated with the patient account, medical questionnaire/OMD, and/or cross-referenced with other patient characteristics.

One or more characteristics (e.g., diagnosis, treatment, symptoms, etc.) of the information received in step 330 may then be determined (step 335). Then, the standardized information received in step 330 and/or the determined characteristics may be associated with the patient account (and thereby associated with other characteristics of the patient) and stored in a database like patient account database 142.

In step 345 (shown in FIG. 3B, which is flowchart showing a continuation of process 300), a request for information regarding the patient may be communicated to an electronic medical record (EMR) database like patient EMR database 130. The request for information may be communicated from a server, like server 102, to the EMR database responsively to receiving a request for information associated with the patient and/or the patient account via, for example, an interface displayed on, for example, user device 124. In some embodiments, the EMR information may be requested on a periodic (e.g., monthly, quarterly, and/or yearly) and/or as-needed (e.g., when an encounter is scheduled and/or treatment is proscribed) basis. In these embodiments, EMR information may be requested in order to, for example, keep a patient account current and updated.

The requested information may then be received from the EMR database (step 350) and translated into a format compatible with the receiving server and/or patient account database such as patient account database 142 (step 355). Execution of step 355 may resemble execution of, for example, step(s) 317 and/or 337. The translated EMR information may then be stored in, or otherwise associated with, a patient account and/or patient characteristics and these association(s), along with the standardized EMR information may be stored in the patient account database (step 360). In some embodiments, execution of step(s) 350 and/or 355 may include associating the received/translated EMR information patient information into a format compatible with the receiving server and/or patient account database such as patient account database 142.

In step 365, a request for information regarding the patient may be communicated to a third-party source of information, such as third-party information source 146, which may be coupled to third-party information database 148. Exemplary third-party information may be from a medical treatment provider not associated with a particular treatment facility associated with a patient account (e.g., a physical therapist, a nutritionist, a radiologist), a pharmacy, a transportation service (which may indicate whether the patient has taken the transportation service to or from a medical appointment), a home healthcare service, a provider of weather information, etc. The request of step 365 may be made in order to, for example, obtain treatment compliance information for a particular patient or group of patients, determine a rate of usage of the services of the third-party, determine whether a patient follow up is needed, determine environmental (e.g., weather, humidity, particulates in the atmosphere, etc.) factors that may, or may not, impact the patient's health, etc.

Then, the requested information may be received from the third-party information source (step 370) and may be translated into the standardized format for association with the patient account and/or storage in the patient account database (step 375). The translation of step 375 may resemble the translations of, for example, step(s) 317, 377, and/or 355. The translated third-party information may then be associated with one or more patient characteristics (e.g., patient identifier, patient demographics, treatment providers, treatment, diagnosis, etc.) and/or patient accounts (step 380) and may be stored in the patient account database (step 385).

In some embodiments, the requests of steps 345 and/or 365 may be performed periodically, as-needed, and/or responsively to a request for information received by, for example, server 102. When performed periodically, the requests may be communicated to the EMR and/or third-party data source on, for example, a daily, weekly, and/or monthly basis. Additionally, or alternatively, the requests of steps 345 and/or 365 may be performed as part of an audit or other review of information associated with one or more patient accounts and/or patient characteristics.

Although presented in a particular order, it is noted that the steps of process 300 may be performed in a different order than what is shown and/or not every step of process 300 may be performed every time process 300 is executed. For example, steps 345-360 and/or 365-385 may not be performed every time process 300 is executed. In another example, steps 365-385 may be performed prior to execution of, for example, step 325.

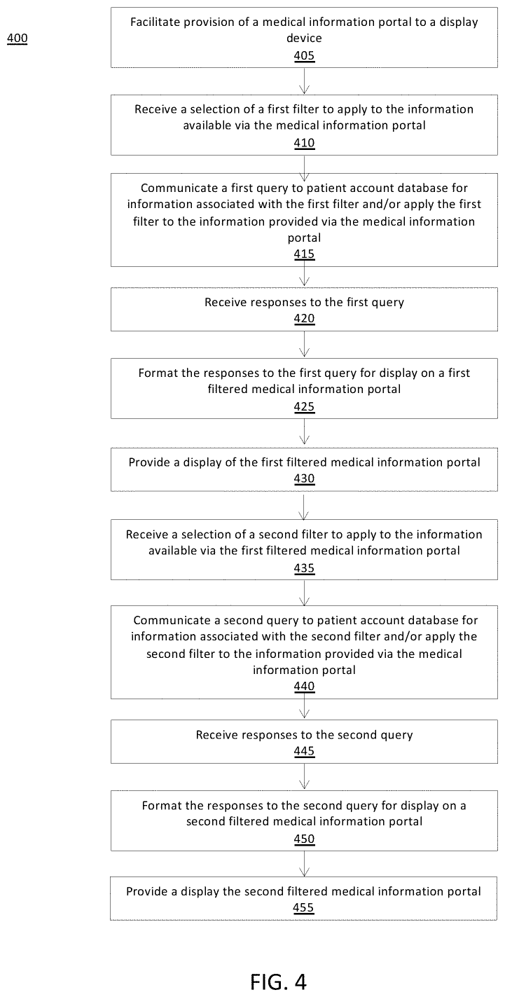

FIG. 4 provides a flowchart illustrating a process 400 for generating and providing for display of one or more medical information portal interface(s) that may be populated with information received from a patient account database responsively to a query for information pertaining to patients who share and/or are associated with one or more characteristics. The information displayed on the medical information portal interfaces may be, for example, wellness and/or improvement scores for patients who share the one or more characteristics. Process 400 may be executed by, for example, systems 100 and/or 101 and/or components thereof.

In step 405, provision of a medical information portal interface may be facilitated by, for example, a server, like server 102, communicating instructions for generating the medical information portal interface to a display device (e.g., display 1112) included a user device like user device 124 and/or a patient device like patient device 128. In some embodiments, the medical information portal interface provided in step 405 may be a home page or initial medical information portal interface page.

In step 410, a selection of a first filter to apply to the information displayed via the medical information portal interface may be received from, for example, an electronic device like user device 124. The first filter may be used to generate a first query that is communicated to a patient account database like patient account database 142 via, for example, a server like server 102 (step 415). The first filter and/or first query may request information for patients/patient accounts associated with one or more characteristics (e.g., diagnosis, a medical questionnaire/OMD taken, a comorbidity, time since treatment began, treatment the patient has, or is, receiving, demographic information, a treatment facility and/or doctor providing care to the patient, etc.).

A set of responses to the first query (also referred to herein as a “first set of responses”) may be received (step 420) and formatted for display (step 425) on a first filtered medical information portal interface that may be provided (step 430) as, for example, a graphic user interface (GUI). The responses to the query may include, for example, wellness scores, improvement scores, patient compliance with treatment information, treatment information, comorbidities, and the like. The responses to the query may be formatted in one or more ways for display on the first filtered medical information portal interface. Exemplary formats include, but are not limited to, graphs like scatter, bar, or pie graphs, lists, letter scores, numerical scores, percentages, histograms, and other representations of the data received in response to the first query.

A first exemplary medical information portal interface home page 501 that may be provided for display via step 430 is provided in FIG. 5A. Medical information portal interface home page 501 includes a first version a first-layer menu 511A by which a user may select one or more categories of filters associated with patient accounts. For example, first-layer menu 511A provides the option (via a drop-down menu (not shown)) of selecting filter categories of a medical facility, a doctor, a team member, and/or a patient associated with patient accounts and associated information to be viewed.

Medical information portal interface home page 501 also includes a first version of a second-layer menu 510A by which a user may select one or more categories of filters to apply to the patient accounts associated with the first-layer menu 511A selections. Exemplary filter categories included in second-layer menu 510A are name and/or type of assessment taken by the patients (referred to as “assessments” in second layer menu 510A), patient ages, patient diagnoses, procedures and/or treatments administered to the patients (referred to as “procedures” on second layer menu 510A), follow up ranges, and a date range for when a treatment may have been administered and/or a duration of time between an initial pre-treatment assessment and one or more post-treatment assessment(s). When a specific filter category included within second-layer menu 510A is not selected, the default action is to not apply any filter pertaining to that particular category to the patient accounts displayed via subsequent interfaces. For example, when a filter for the assessment's category is not selected or otherwise set, patient accounts displayed in subsequent interfaces will not be filtered by assessment type or category and, as such, patient accounts associated with all types of assessments may be displayed in response to a query of a patient account database that is responsive to the first and/or second menu options selected.

Medical information portal interface home page 501 also includes a top menu that includes the following options: dashboard, assessments, patients, and view of which view has been selected and a corresponding drop-down menu 520 both different views available to the user (in this case Dr. Saliman) is provided. Drop down menu 515 includes plurality of facility views that includes a first facility (Hospital A) and a second facility (specialty clinic) and a plurality of layout views that includes a doctor view and a team view. Of these options, the Hospital A facility view, and doctor layout view have been selected and the center menu option of first version a first-layer menu 511A is set to the Hospital A facility.

FIG. 5B provides a second exemplary medical information portal interface home page 502 with a second version of a first-layer menu 511B and a second version of a second-layer menu 510B that may be provided for display in step 405. Second version of first-layer menu 511B provides three filter categories: center, provider, and patient. Second version of second-layer menu 510B provides filters for assessments, diagnosis, medication, procedures, procedure sub filter, time post procedure, and age range. Second version of second-layer menu 510B also provides an option to select and/or add on additional filters that may be used to build a query of patent account database 142. Second exemplary medical information portal interface home page 502 further includes a list of tabs 525, the selection of which may cause display of an associated interface/page as described herein. Tabs provided in list of tabs 525 include an outcome results overview tab, a notification tab a recent results summary tab, a may need attention tab, and an incomplete follow-up tab. Filters and/or categories selected from first and/or second-layer menus 511A, 511B, 510A, and/or 510B may be applied to information displayed on one or more of the pages accessed via selection of a corresponding tab from list 525.

FIG. 5C provides a third exemplary medical information portal interface home page 503 wherein second version of a first-layer menu 511B has different values for the number of centers, providers, and patients than the second version of first-layer menu 511B of medical information portal interface home page 502 but is otherwise similar. Third exemplary medical information portal interface home page 503 has a third version of a second-layer menu 510C that may be provided for display in step 405. Third version of second-layer menu 510C provides the filtering options for assessments, diagnosis, medication, procedures, age range, patient-reported compliance, adverse life events, confounding injuries, and ad hoc filters. Third exemplary medical information portal interface home page 503 further includes list of tabs 525.

FIG. 5D provides a screen shot of an exemplary medical information portal interface 504 that includes an outcome results overview window 535 and a fourth second-layer menu 510D that includes filters for assessments, diagnosis, medications, procedures, age range, and patient-reported compliance. Interface 504 may be displayed and/or outcome results overview window 535 may be displayed responsively to a user's selection of the outcome results overview tab from list of tabs 525. The information displayed on outcome results overview window 535 is information pertaining to patient outcomes for the center, providers, and patients selected from first layer menu 511B when no additional filters from second-layer menu 510 are applied to patient account data (in the form of wellness and improvement scores) are queried for, and received from, the patient account database via medical information portal interface home page 504. Thus, the information provided by outcome results overview window 535 pertains to outcomes for the widest population of patients associated with the center and/or providers using the medical information portal interfaces disclosed herein.

Outcome results overview window 535 includes a bar graph 508 that shows an average initial wellness score for all patients (in this case, 38), an average follow up wellness score for all patients (in this case, 62), and an average overall improvement score, or percentage (in this case, 39%). Outcome results overview window 535 also includes a set of improvement statistics or percentages 509 that, in the example of outcome results overview window 535 provides a number and a percentage patients who have improved by at least 20 percent since an initial (typically pre-treatment) communication of a medical questionnaire (or group of medical questionnaires), a number and percentage of all patients who have improved by at least 50 percent since the initial communication of the medical questionnaire, and a number and percentage of all patients whose condition has worsened since an initial communication of the medical questionnaire.

FIGS. 5E-5G provide medical information portal interface home page 505, 506, and 507, respectively. Each of these medical information portal interface home pages include an expanded dropdown list of assessment types a user may select from when querying the patient account database for information about patients who took the respective assessment(s). More particularly, FIG. 5E provides a fifth exemplary medical information portal interface home page 505 with an expanded drop-down menu 530A that displays a plurality of assessment filtering options a user may select from with some assessment types/categories falling under a heading. FIG. 5F provides a sixth exemplary medical information portal interface home page 506 with an expanded drop-down menu 530B that displays a plurality of assessment filtering options a user may select from wherein an assessment pertaining to anxiety and atrial fibrillation are selected. FIG. 5G provides a seventh exemplary medical information portal interface home page 507 with an expanded drop-down menu 530C that displays a plurality of assessment filtering options a user may select from, wherein a user has selected a filter pertaining to shoulder assessments.

Each of the plurality of assessment filtering options provided by expanded drop-down menus 530A, 530B, and/or 530C may be associated with one or more medical questionnaires, treatments, and/or procedures and this association may be in the form of, for example, one or more codes that are in a standardized format compatible with the patient account database and/or in a format that may be inserted into a query of the patient account database upon selection by a user. For example, when a user selects an assessment type from expanded drop-down menu 530A, a query of the patient account database may be generated that uses one or more codes or identifiers associated with the selected assessment type so that a processor may search the patient accounts stored in patient account database for patient accounts associated with the code or identifier for the selected assessment type.

FIG. 5H provides an eighth exemplary medical information portal interface home page 508 with second version of a first-layer menu 511B, a fifth version of a second-layer menu 510E, and a truncated list 525 that may be displayed following receipt of a first filter in step 410. Fifth version of second-layer menu 510E provides filters for assessments, diagnosis, medication, procedures, procedure sub filters, time post procedure, age range, patient reported compliance, ad hoc filters, and adverse life event filters. Of these filters, the assessments filter has been set to shoulder via, for example, a drop-down menu like drop-down menu 530A, 530B, and/or 530C, the age range has been set to 25-80 years, and the patient reported compliance has been set to range between 70%-100%.

FIG. 5I provides a screen shot of a ninth exemplary medical information portal interface home page 509 that shows a first filter selection of shoulder assessment where the tab “incomplete follow ups” from list 525 has been selected so that patients who have not yet completed their follow up shoulder assessment are listed in an incomplete follow ups window 540. The user may then access more information about each of the patients with an incomplete assessment/OMD/medical questionnaire and select an icon that effectuates re-sending of the assessment to the patient via, for example, a computing device used by the patient.

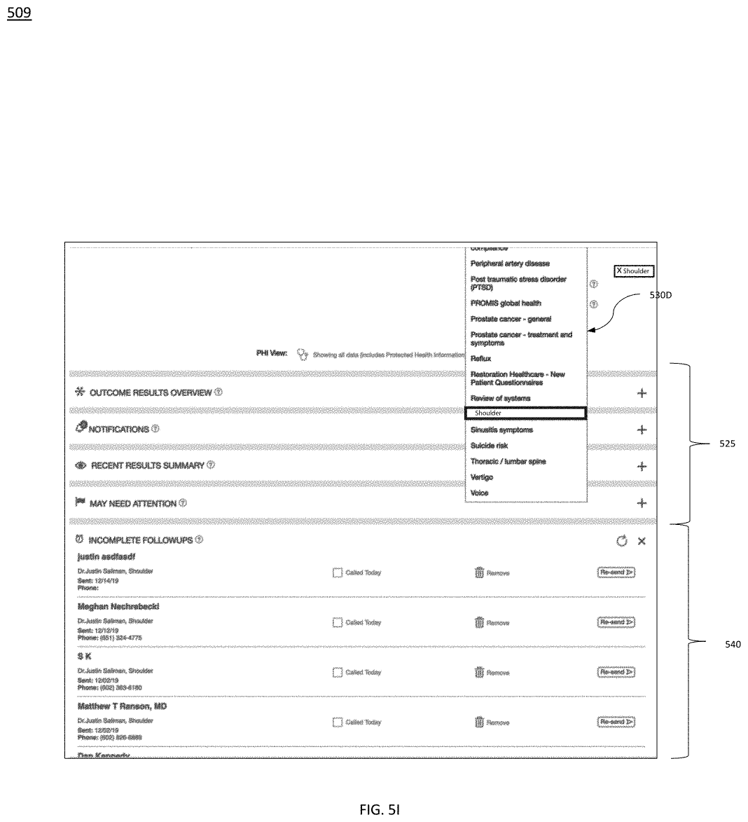

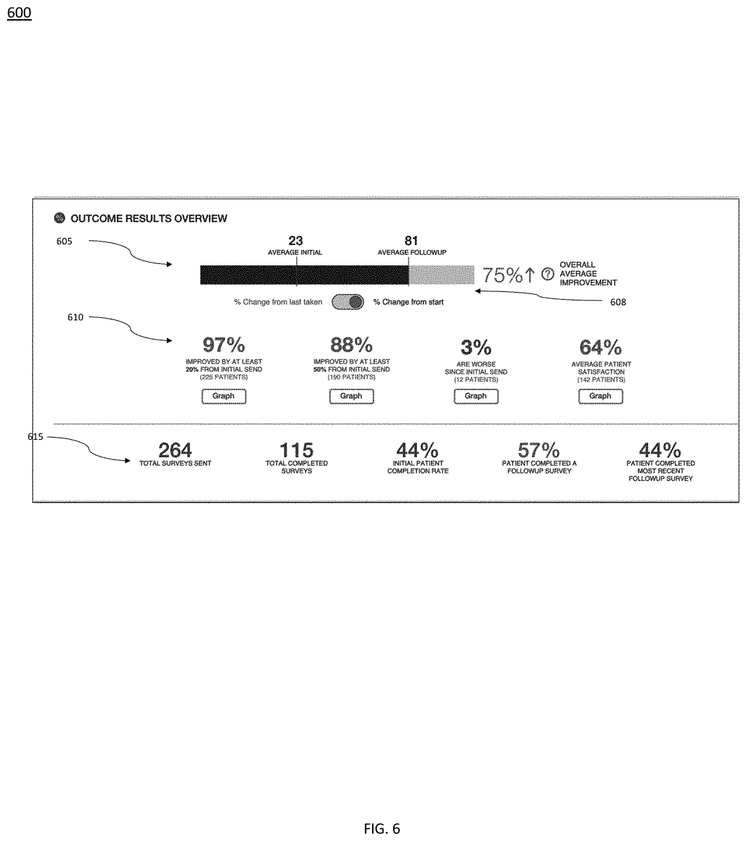

FIG. 6 provides an exemplary outcome results overview interface page 600 that may be provided for display via execution of step 430. The information displayed on outcome results overview page may correspond to, for example, wellness and/or improvement scores that are associated with patients, via their respective patient accounts and/or characteristics associated with the respective patient accounts, who meet the requirements/contents of the first query. Outcome results overview interface page 600 shows formatted responses to the first query in the form of a bar graph 605 that shows an average initial wellness score (in this case, 23) an average follow up wellness score (in this case, 81), and an average overall improvement score (in this case, 75). Outcome results overview interface page 600 also includes a toggle button 608 that allows the user to toggle between a view of improvement scores that shows a percentage change in wellness score from either 1) last taken or 2) change from start (e.g., a wellness score determined using responses to a medical questionnaire received prior to a treatment. Outcome results overview interface page 600 further shows formatted responses to the first query in the form of an outcome results row 610 that shows statistics for improvement of patients from an initial determination and an average rate of satisfaction. Outcome results overview interface page 600 further shows formatted responses to the first query in the form of a survey statistics row 615 that shows how may surveys (medical questionnaires and/or OMDs) were sent to patients and completed by the patients.

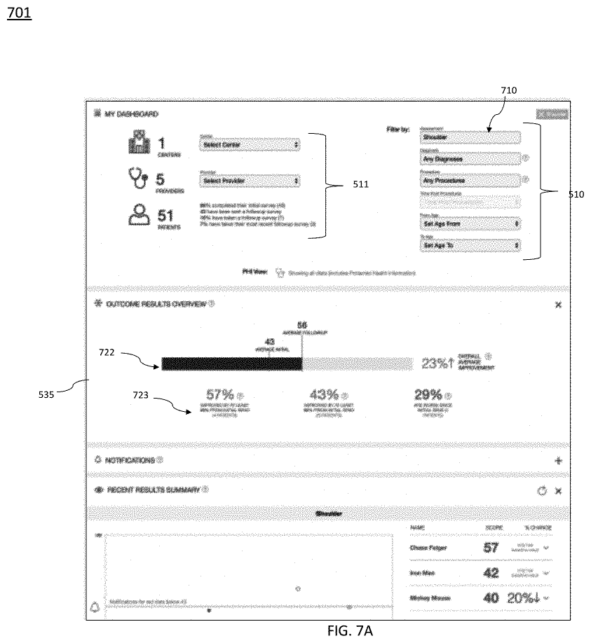

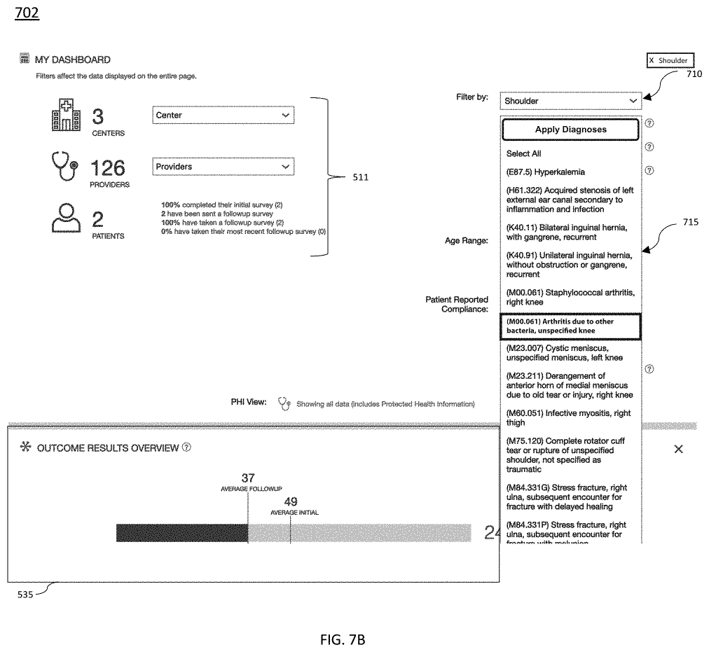

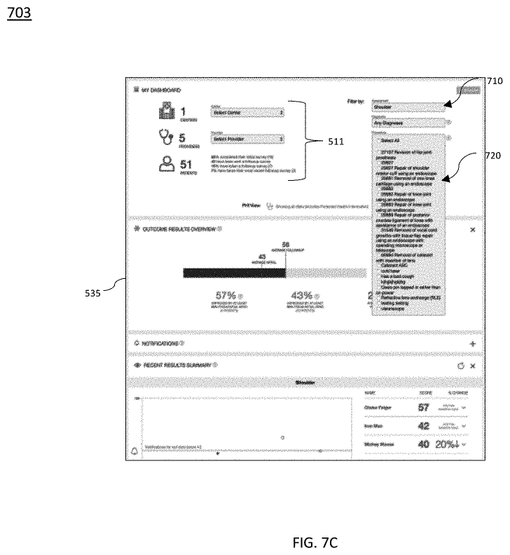

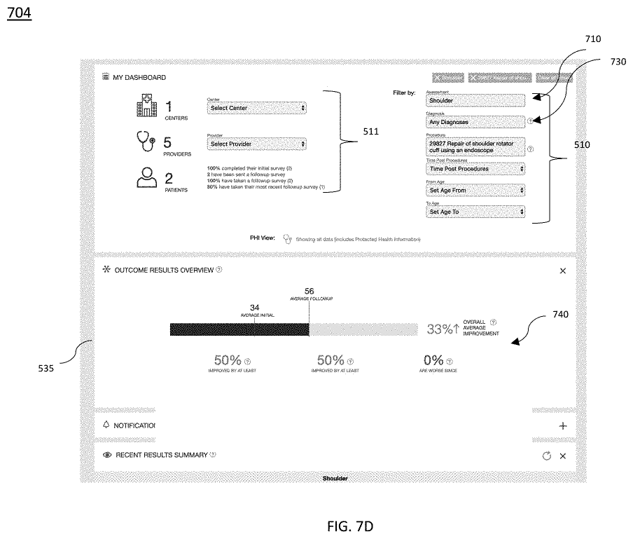

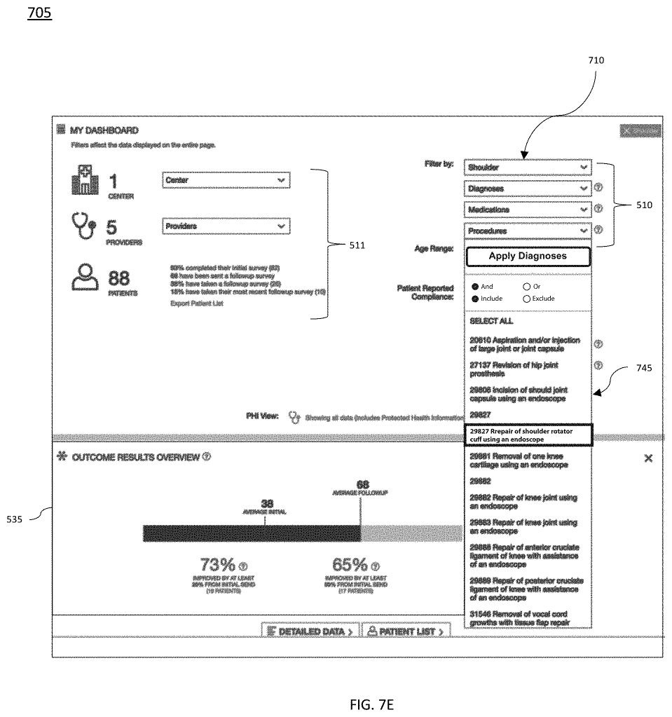

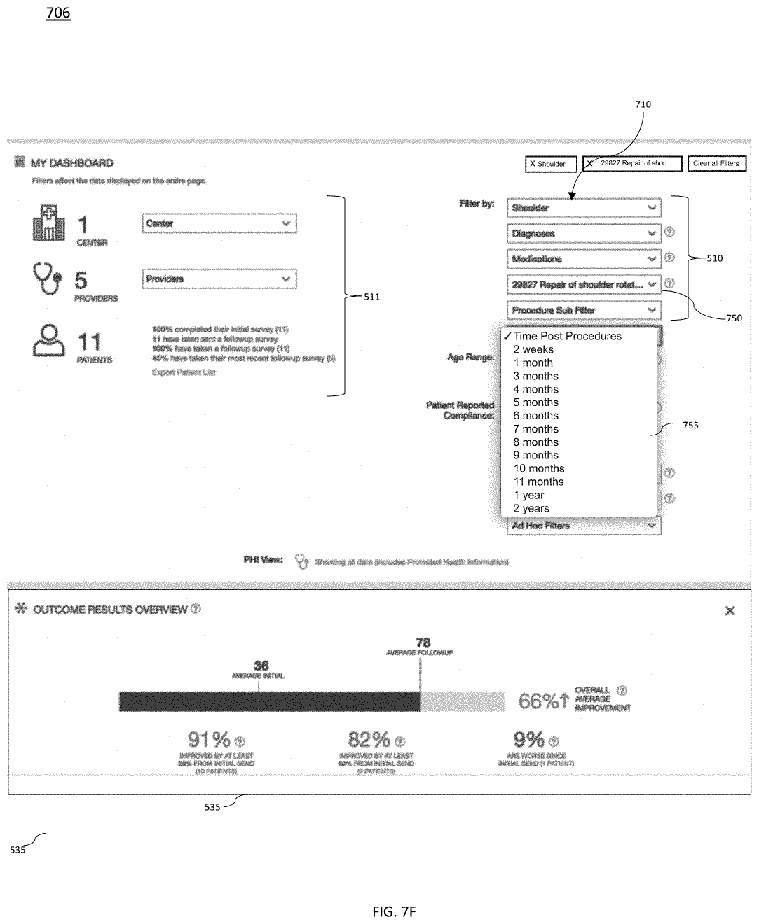

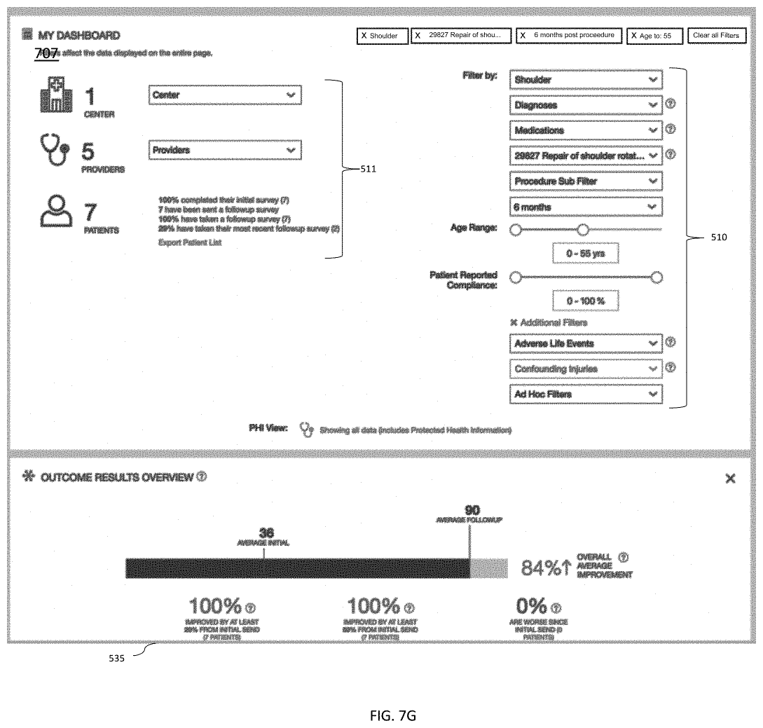

FIG. 7A provides an exemplary medical information portal interface home page 701 that includes an outcome results overview interface window 535 that may be provided for display via execution of step 430 when the first selected filter of second-layer menu 510 is for shoulder assessments as shown in text box 710. Outcome results overview interface window 535 includes a bar graph 722 that shows an average initial wellness score for shoulder assessments (in this case, 43), an average follow up wellness score for shoulder assessments (in this case, 56), and an average overall improvement score, or percentage (in this case, 23%). Outcome results overview interface window 535 also includes an outcome results row 723 that shows statistics for improvement (i.e., change of wellness score over time) of patients from an initial determination and an average rate of satisfaction.

In step 435, a selection of a second filter to apply to the information available for display on the first filtered medical information portal interface may be received. This selection may be a selection of one or more of the options provided by, for example, first-layer menu 511, second-layer menu 510, and/or dropdown menu 515. Often times, the second filter modifies, or narrows, the patient account information extracted from the patient account database and, as such, is frequently a filter selected from one of the options provided by second-layer menu 510 when a first option has been selected from second-layer menu 510 as well. For example, if a user selected a medical center and/or provider from first-layer menu 511, and also selected a second filter in the form of an assessment type, then the results shown on medical information portal interface home page 701 would be filtered by the selected medical center and/or provider from first-layer menu 511 and also a second filter selected from, for example, second-layer menu 510, and/or dropdown menu 515.

FIG. 7B shows a second exemplary medical information portal interface home page 702 for selecting a second filter to apply to the information available via a first medical information interface like medical information interface 701. Second exemplary medical information portal interface home page 702 includes an expanded dropdown menu 715 for selecting a diagnosis to be filtered for along with the shoulder assessments selection shown in text box 710. In the example of FIG. 7B, drop-down menu shows that diagnosis (M00,869) arthritis due to other bacterial, unspecified knee is selected (as shown because of the color highlighting of this option) has been selected.

FIG. 7C shows a third exemplary medical information portal interface home page 703 for selecting a second filter to apply to the information that may be selected from an expanded drop-down menu 720 for selecting a procedure to be filtered for along with the shoulder assessments selection shown in text box 710.

In step 440, a second query for information associated with the second filter may be communicated to the patient account database. Responses to the second query may be received (step 445). Additionally, or alternatively, execution of step 440 may include application of the second filter to the first set of responses received in step 420, thereby generating a modified set of first responses, so that responses to the first query that do not match the second query are removed from the first set of query results to generate the second set of query results (step 445). The received responses to the second query and/or modified set of first responses may then be formatted for display on a second filtered medical information portal page (step 450). The second filtered medical information portal page may then be provided to display device for display to a user (step 455) on an interface like interface 704 shown in FIG. 7D, which shows the that first selected filter is for shoulder assessments as shown in text box 710 and the second selected filter, selected from expanded drop-down menu 720, is a procedure, namely the repair of a shoulder rotator cuff using an endoscope.

FIGS. 7E-7G provide screen shots of exemplary medical information portal interfaces 705, 706, and 707 whereby a user has successively applied multiple filters to the data that is queried for from the patient account database. In interface 705 of FIG. 7E, a first filter of shoulder assessments and the outcome results overview window 535 of FIG. 7E shows that patients with patient accounts in patient account database matching the first query (i.e., patient who took a shoulder assessment) had an initial wellness score of 38 and an average follow up wellness score of 68 along with statistics showing 73% of patients improved at least 20% from an initial assessment and 65% of patients improved at least 50% from an initial assessment.

Interface 705 of FIG. 7E also provides a mechanism for a user to select a second filter, or second queried-for characteristic, in the form of a drop down menu 745 for the selection of a procedure and/or treatment. In the case of FIG. 7E, the user has selected a procedure corresponding to procedure code 29827 repair of shoulder rotator cuff using an endoscope as is shown by the highlighting of this procedure from drop down menu 745 and the patient data queried for from patient accounts stored on the patient account database responsively to these two filters is displayed in the outcome results overview window 535 of FIG. 7E (step 455).

Interface 706 shows application of the second filter for procedure code 29827 repair of shoulder rotator cuff using an endoscope (shown in text box 750) to data filtered using the shoulder assessment filter as seen in the updated values of outcome results overview window 535, which shows an initial score of 38 and an average follow up score of 78 along with statistics showing 91% of patients improved at least 20% from an initial assessment and 82% of patients improved at least 50% from an initial assessment, an overall average improvement of 66% and 9% of patients are worse since the initial assessment when the shoulder assessment and the procedure code 29827 repair of shoulder rotator cuff using an endoscope filters are applied to the patient data. In addition, interface 706 provides a mechanism for the user to apply a third filter in the form of a drop down menu 755 for selecting a time period post procedure (in this case, 6 months).

Interface 707 of FIG. 7G shows application of a third filter for a time period of six months post procedure to data filtered using the shoulder assessment and 29827 repair of shoulder rotator cuff using an endoscope filters as seen in the updated values of outcome results overview window 535, which shows an initial score of 38 and an average follow up score of 90 along with statistics showing 100% of patients improved at least 20% from an initial assessment and 100% of patients improved at least 50% from an initial assessment, an overall average improvement of 84% and 0% of patients are worse since the initial assessment when the shoulder assessment, the procedure code 29827 repair of shoulder rotator cuff using an endoscope, and six months post procedure filters are applied to the patient data. In addition, interface 707 provides the user with a mechanism to select additional filters to apply to the data in the form of patient reported compliance, adverse life events, confounding injuries, and ad hoc filters.

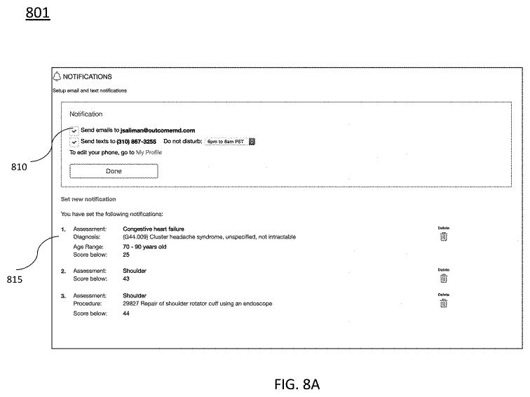

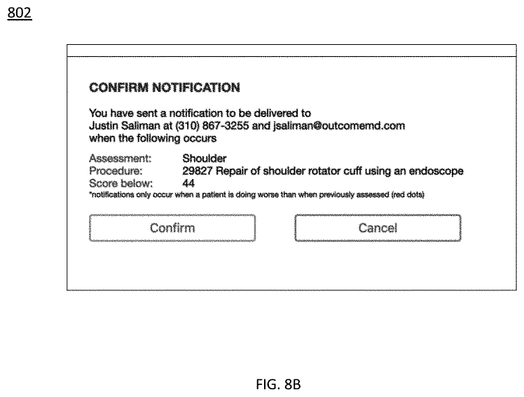

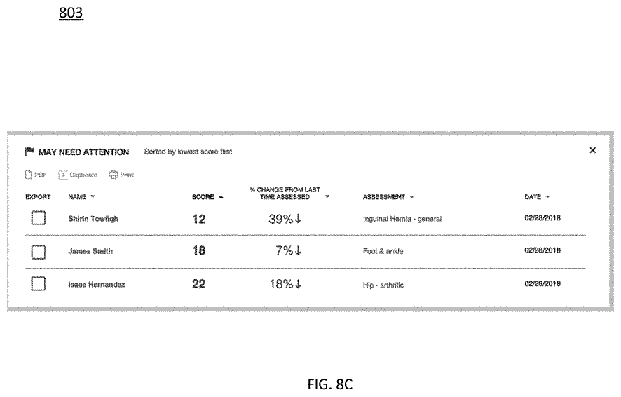

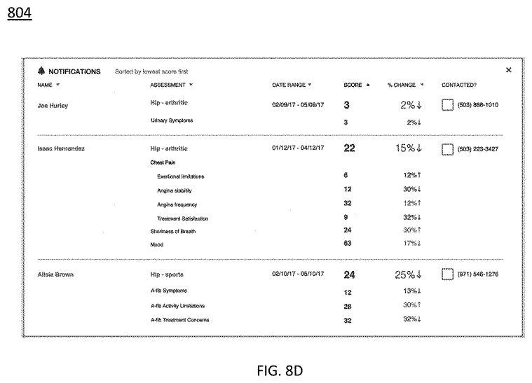

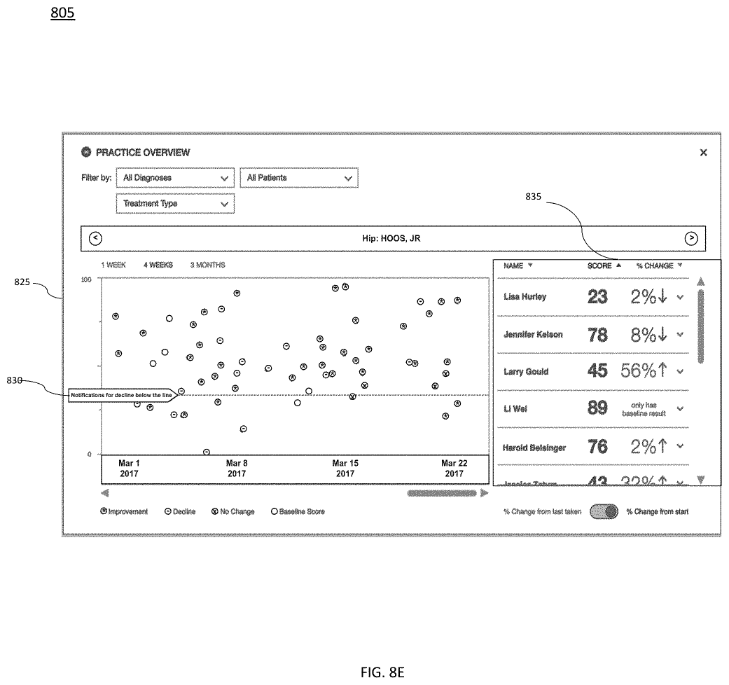

FIG. 8A provides the screen shot of a notification setting interface 801 by which a user may set one or more criteria for sending a notification to, for example, the user, another person (e.g., physician, nurse, caregiver, etc.) and/or system (e.g., central nurse's station computer, emergency services, etc.). Notification setting interface 801 has a notification communication method window 810 wherein a user may select and/or enter information regarding how the notified party is to be notified. For example, notification communication method window 810 provides the option to send the notified party an email and/or an SMS text message and also provides options for selecting when, during the day, a notification is to be sent to the notified party. Notification setting interface 801 also has a notification criteria selection window 815 that provides a number of options for the selection of various criteria to use when determining whether or not to send a notification. For example, notification criteria selection window 815 includes notification criteria for various assessments, in this case notifications are set for a congestive heart failure assessment with a diagnosis of 944-069 congestive heart failure syndrome, with an age range between 70-90 years old and a wellness score of below 25, a shoulder assessment with a wellness score of below 43, and a shoulder assessment with the procedure corresponds to procedure number 29827 for repair of shoulder rotator cuff using an endoscope with a wellness score of below 44. FIG. 8B provides a confirmation window 802 that may be used to confirm information entered into notification setting interface 801.

FIG. 8C provides an exemplary notification interface 803 that provides a list of individuals meeting the criteria for the communication of a notification as may be set via, for example, notification setting interface 801. The list of individuals may include, for example, a name of individuals meeting the criteria for the communication of notification along with information about each of these individuals; in this case, a wellness score, a percentage change from the last time the assessment was performed, an assessment name, and a date of the list assessment.

FIG. 8D provides another exemplary notification interface 804 that provides a list of individuals meeting the criteria for the communication of a notification as may be set via, for example, notification setting interface 801. The list of individuals may include, for example, a name of an individual meeting the criteria for the communication of notification along with information about each of these individuals; in this case, a wellness score for different assessments, a percentage change from the last time the assessment was performed, an assessment name, a date of the last assessment, and a box by which a user may indicate that the patient has been contacted.

FIG. 8E provides an interface 805 with an exemplary scatter-plot graph of wellness scores for a plurality of patients 825 along with a visual notification setting graphic element 830 and a list of patients 835 and their corresponding wellness scores and percentage changes in wellness scores when compared to a prior-determined wellness score. Scatter plot graph 825 provides a graph of wellness scores for patients over time, in this instance the time period between Mar. 1, 2017 and May 22, 2017. The icons used to represent wellness scores for patients may be encoded with color (e.g., green showing an improvement between a prior-answered assessment and a currently-answered assessment, red showing an decline between a prior-answered assessment and a currently-answered assessment, yellow showing no change between a prior-answered assessment and a currently-answered assessment) and/or a shape of an icon on the graph (e.g., an empty circle showing a baseline wellness score determination, a star showing an improvement between a prior-answered assessment and a currently-answered assessment, a filled in circle showing an decline between a prior-answered assessment and a currently-answered assessment, and/or a square showing no change between a prior-answered assessment and a currently-answered assessment). A user may adjust a notification threshold by adjusting a position (e.g., moving up or down) of visual notification setting graphic element 830 within graph 825 to set one or more wellness score thresholds so that a wellness score below the threshold will trigger communication of a notification.

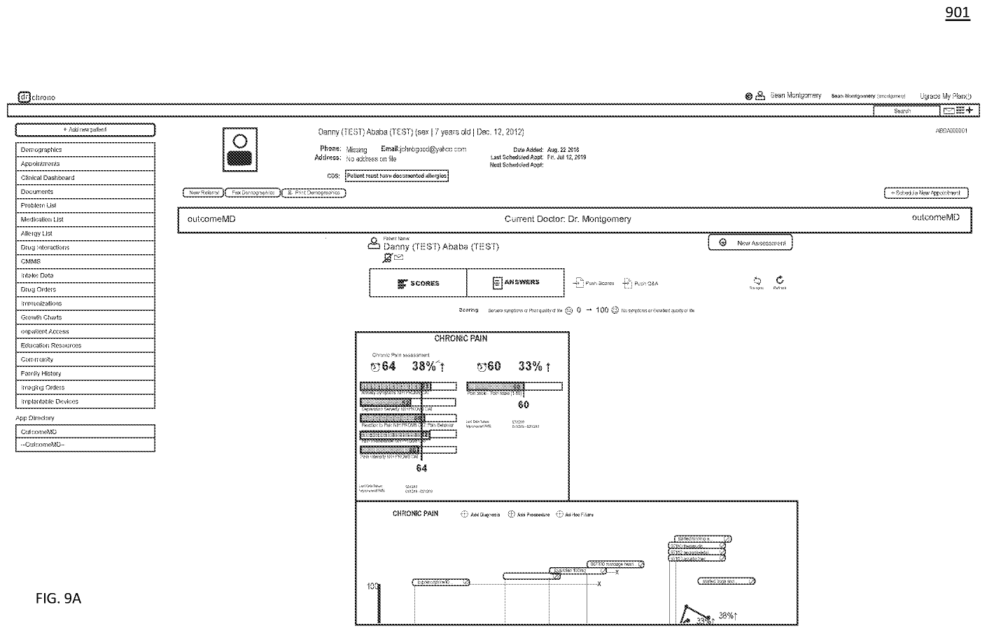

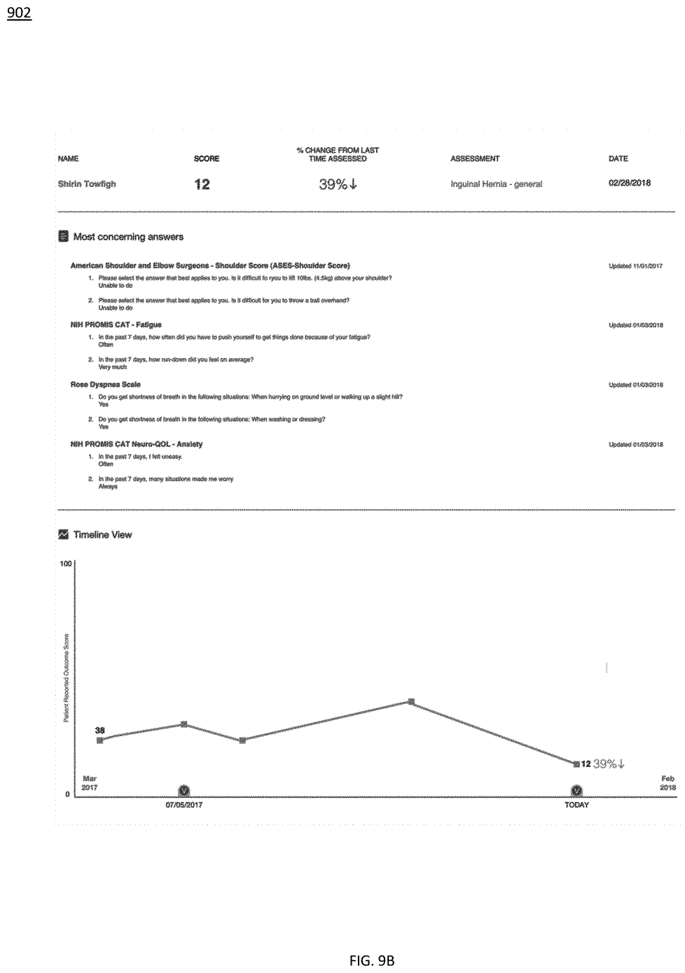

FIG. 9A provides an exemplary patient-specific interface 901 that shows patient-identifying information (e.g., name, phone, etc.) along with information regarding the patient's medical treatment such as the patient's doctor, last appointment, etc. Patient-specific interface 901 also includes information and wellness scores for a test case named Danny (TEST) Ababa (TEST) for chronic pain assessments. FIG. 9B provides another exemplary patient-specific interface 902 that shows patient-identifying information (e.g., name, phone, etc.) along with information regarding the patient's wellness scores for a plurality of assessments. Interface 902 also shows a line graph of the patient's wellness scores over time from March 2017 to February 2018. Interfaces 901 and/or 902 may be provided to a user responsively to a request/query. Additionally, or alternatively, a user may input information into a patient account via entry of information into interfaces 901 and/or 902.

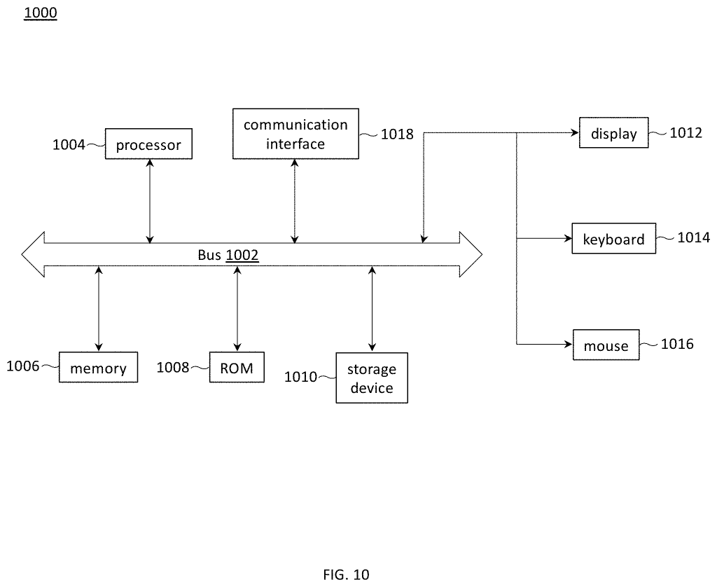

FIG. 10 is a block diagram showing a system 1000 includes a bus 1002 or other communication mechanism for communicating information, and a processor 1004 coupled with the bus 1002 for processing information. Computer system 1000 also includes a main memory 1006, such as a random-access memory (RAM) or other dynamic storage device, coupled to the bus 1002 for storing information and instructions to be executed by processor 1004. Main memory 1006 also may be used for storing temporary variables or other intermediate information during execution of instructions to be executed by processor 1004. Computer system 1000 further includes a read only memory (ROM) 1008 or other static storage device coupled to the bus 1002 for storing static information and instructions for the processor 1004. A storage device 1010, for example a hard disk, flash memory-based storage medium, or other storage medium from which processor 1004 can read, is provided and coupled to the bus 1002 for storing information and instructions (e.g., operating systems, applications programs and the like).

Computer system 1000 may be coupled via the bus 1002 to a display 1012, such as a flat panel display, for displaying information to a computer user. An input device 1014, such as a keyboard including alphanumeric and other keys, may be coupled to the bus 1002 for communicating information and command selections to the processor 1004. Another type of user input device is cursor control device 1016, such as a mouse, a track pad, or similar input device for communicating direction information and command selections to processor 1004 and for controlling cursor movement on the display 1012. Other user interface devices, such as microphones, speakers, etc. are not shown in detail but may be involved with the receipt of user input and/or presentation of output.

The processes referred to herein may be implemented by processor 1004 executing appropriate sequences of computer-readable instructions contained in main memory 1006. Such instructions may be read into main memory 1006 from another computer-readable medium, such as storage device 1010, and execution of the sequences of instructions contained in the main memory 1006 causes the processor 1004 to perform the associated actions. In alternative embodiments, hard-wired circuitry or firmware-controlled processing units may be used in place of or in combination with processor 1004 and its associated computer software instructions to implement the invention. The computer-readable instructions may be rendered in any computer language.

In general, all of the above process descriptions are meant to encompass any series of logical steps performed in a sequence to accomplish a given purpose, which is the hallmark of any computer-executable application. Unless specifically stated otherwise, it should be appreciated that throughout the description of the present invention, use of terms such as “processing”, “computing”, “calculating”, “determining”, “displaying”, “receiving”, “transmitting” or the like, refer to the action and processes of an appropriately programmed computer system, such as computer system 1000 or similar electronic computing device, that manipulates and transforms data represented as physical (electronic) quantities within its registers and memories into other data similarly represented as physical quantities within its memories or registers or other such information storage, transmission or display devices.

Computer system 1000 also includes a communication interface 1018 coupled to the bus 1002. Communication interface 1018 may provide a two-way data communication channel with a computer network, which provides connectivity to and among the various computer systems discussed above. For example, communication interface 1018 may be a local area network (LAN) card to provide a data communication connection to a compatible LAN, which itself is communicatively coupled to the Internet through one or more Internet service provider networks. The precise details of such communication paths are not critical to the present invention. What is important is that computer system 1000 can send and receive messages and data through the communication interface 1018 and in that way communicate with hosts accessible via the Internet. It is noted that the components of system 1000 may be located in a single device or located in a plurality of physically and/or geographically distributed devices.

We claim:

1. A method comprising:
providing, by a processor, a medical questionnaire to a patient associated with a patient account that is associated with a plurality of characteristics for the patient, the medical questionnaire pertaining to at least one of a diagnosis for the patient and a treatment received by the patient;
receiving, by the processor, a set of responses to the medical questionnaire from the patient;
determining, by the processor, a wellness score for the patient using the set of responses;
translating, by the processor, the set of responses and the wellness score into a standardized format compatible with a patient account database thereby generating a standardized set of responses and a standardized wellness score;
associating, by the processor, the standardized set of responses and the standardized wellness score with the patient account and the plurality of characteristics for the patient;
saving, by the processor, the standardized set of responses, the standardized wellness score, and the associations between the standardized set of responses and the standardized wellness score with the patient account and at least one of the plurality of characteristics for the patient in the patient account database;
querying, by the processor, an electronic medical record database for information about the patient;
receiving, by the processor, electronic medical record information about the patient from the electronic medical record database responsively to the query;
translating, by the processor, the received electronic medical record information about the patient into the standardized format thereby generating a standardized set of electronic medical information;
associating, by the processor, the standardized set of electronic medical record information with the patient account and the plurality of characteristics of the patient;
saving, by the processor, the standardized set of electronic medical record information and the association between the standardized set of electronic medical record information and patient account in the patient account database; and
providing, by the processor, a user access to the patient account database via a display device.

2. The method of claim 1, further comprising:
receiving, by the processor, patient information from a medical professional;
translating, by the processor, the patient information into the standardized format thereby generating a standardized set of patient information;
associating, by the processor, the standardized set of patient information with the patient account; and
saving, by the processor, the standardized set of patient information and the association between standardized set of patient information with the patient account in the patient account database.

3. The method of claim 2, wherein the patient information is at least one of demographic information about the patient, a diagnosis of the patient, a treatment administered to the patient, a comorbidity of the patient, and a degree of the patient's compliance with a treatment.

4. The method of claim 1, wherein translating at least one of the set of responses, the wellness score, and the received electronic medical record information about the patient into the standardized format includes:
analyzing, by the processor, at least one of the set of responses, the wellness score, and the received electronic medical record information about the patient to determine a characteristic of the at least one set of responses, wellness score, and received electronic medical record information about the patient; and
assigning, by the processor, a code to the at least one set of responses, wellness score, and received electronic medical record information about the patient responsively to the determined characteristic of the at least one set of responses, wellness score, and received electronic medical record information about the patient, wherein the assigning includes associating the assigned code with the at least one set of responses, wellness score, and received electronic medical record information and the saving includes saving the assigned code with the at least one set of responses, wellness score, and received electronic medical record information.

5. The method of claim 1, wherein translating at least one of the set of responses, the wellness score, and the received electronic medical record information about the patient into the standardized format includes removing any patient-identifying information from the at least one of the set of responses, the wellness score, and the received electronic medical record information about the patient.

6. The method of claim 1, wherein each of the plurality of characteristics are associated with a code and the association of the plurality of characteristics with the standardized set of responses and the standardized wellness score with the patient account is performed by associating a code for each respective characteristic with the standardized set of responses and the standardized wellness score.

7. The method of claim 1, wherein the patient account database stores patient accounts for at least one thousand patients, each of the patient accounts being associated with a patient, a set of characteristics for each respective patient, and wellness scores determined for each respective patient, the method further comprising:
receiving, by the processor, a request for wellness scores of patients who match a specified characteristic;
querying, by the processor, the patient account database for wellness scores of patients who match the specified characteristic responsively to the received request;
receiving, by the processor, a set of wellness scores of patients who match the specified characteristic responsively to the query;
formatting, by the processor, the set of wellness scores for display on the display device; and
providing, by the processor, the formatted set of wellness scores to the display device.

8. The method of claim 7, wherein the request is a first request, the specified characteristic is a first specified characteristic, and the set of wellness scores is a first set of wellness scores, the method further comprising:
receiving, by the processor, a second request for wellness scores associated with patients that are associated to a second characteristic;
querying, by the processor, the patient account database for wellness scores of patients associated with the second specified characteristic responsively to the second request;
receiving, by the processor, a second set of wellness scores from the patient account database that match the second characteristic responsively to the query for wellness scores of patients associated with the second specified characteristic;
formatting, by the processor, the second set of wellness scores for display on the display device; and
providing, by the processor, the formatted second set of wellness scores to the display device.

9. The method of claim 1, wherein the set of responses is a first set of responses and the wellness score is a first wellness score, the method further comprising:
subsequently providing, by the processor, the medical questionnaire to the patient;
receiving, by the processor, a second set of responses to the medical questionnaire from the patient;
determining, by the processor, a second wellness score for the patient using the second set of responses;
determining, by the processor, an improvement score for the patient by comparing the first and second wellness scores;
translating, by the processor, the second set of responses, the second wellness score, and the improvement score the standardized format thereby generating a second standardized set of responses, a second standardized wellness score, and a standardized improvement score;
associating, by the processor, the second standardized set of responses, the second standardized wellness score, and the standardized improvement score with the patient account and the plurality of characteristics for the patient; and
saving, by the processor, the standardized second set of responses, the standardized second wellness score, the standardized improvement score, and the associations between the standardized second set of responses, the standardized second wellness score, the standardized improvement score with the patient account and the plurality of characteristics for the patient in the patient account database.

10. The method of claim 9, wherein improvement scores have been determined for at least one thousand patients, the method further comprising:
receiving, by the processor, a request for improvement scores for patients that are associated with a specific characteristic;
querying, by the processor, the patient account database for improvement scores of patients associated with the specific characteristic responsively to the request;
receiving, by the processor, a set of improvement scores from the patient account database that match the specific characteristic responsively to the query;
formatting, by the processor, the set of improvement scores for display on the display device; and
providing, by the processor, the formatted set of improvement scores to the display device.

11. The method of claim 9, wherein improvement scores have been determined for at least one thousand patients, the method further comprising:
receiving, by the processor, a subsequent request for improvement scores associated with patients that are associated to at least two specific characteristics;
querying, by the processor, the patient account database for improvement scores of patients associated with the at least two specific characteristics responsively to the subsequent request;
receiving, by the processor, a set of improvement scores from the patient account database that match the at least two specific characteristic responsively to the query;
formatting, by the processor, the set of improvement scores for display on the display device; and
providing, by the processor, the formatted set of improvement scores to the display device.

12. The method of claim 1, further comprising:
communicating, by the processor, a request for information to a third party source of information, the third party source of information not including the patient, a treatment provider for the patient, and the patient account database;
receiving, by the processor, a set of third party information from the third party source of information responsively to the request;
translating, by the processor, the set of third party information into the standardized format thereby generating a standardized set of third party information;
associating, by the processor, the standardized set of third party information with the patient account; and
saving, by the processor, the standardized set of third party information and the association between standardized set of third party information with the patient account in the patient account database.

13. A method comprising:
receiving, by a processor, a request for wellness scores associated with patients that are associated with a characteristic;
querying, by the processor, a patient account database for wellness scores of patients associated with the characteristic, the patient account database storing patient account information for at least one thousand patients, each patient account being associated with a patient and at least one wellness score determined for the patient responsively to receiving a set of responses to a medical questionnaire from the patient;
receiving, by the processor, a set of wellness scores that match the characteristic from the patient account database responsively to the query;
formatting, by the processor, the set of wellness scores for display on a display device; and
providing, by the processor, the formatted set of wellness scores to the display device.

14. A method of claim 13, wherein the formatting of the set of wellness scores comprises calculating an average wellness score for all patients who match the characteristic.

15. The method of claim 13, wherein the characteristic is at least one of demographic information of the patients, a diagnosis of the patients, a treatment administered to the patient, and a time following treatment being administered to the patient.

16. The method of claim 13, wherein the query further includes a request for improvement scores associated with the patients who are associated to the characteristic, an improvement score being determined using two wellness scores for a patient determined at different points of time.

17. The method of 15, the query further including a duration of time following initiation of a treatment administered to the patient, wherein a duration of time between an initially-determined and later-determined wellness score for the corresponds to the duration of time.

18. The method of claim 13, wherein the request is a first request, the characteristic is a first characteristic, and the set of wellness scores is a first set of wellness scores, the method further comprising:
receiving, by the processor, a second request for wellness scores associated with patients that are associated to a second characteristic;
querying, by the processor, the patient account database for wellness scores of patients associated with the second characteristic responsively to the second request;
receiving, by the processor, a second set of wellness scores from the patient account database that match the second characteristic responsively to the query;
formatting, by the processor, the second set of wellness scores for display on a display device; and
providing, by the processor, the formatted second set of wellness scores to the display device.

19. The method of claim 17, wherein the second request narrows a scope of the first request.

20. The method of claim 17, wherein formatting the second set of wellness scores comprises calculating an average wellness score for all patients who match the first and second characteristics.
